(19) United States
(12) Patent Application Publication
(10) Pub. No.: US 2013/0115257 A1
(43) Pub. Date: May 9, 2013

(54) COMPOSITIONS AND METHODS FOR REMOVAL OR DESTRUCTION OF AMYLOID FIBRIL OR AMYLOID ADHESIN COMPRISING AGGREGATES

(71) Applicants: Maarten Gysemans, Schriek (BE); Johan Snauwaert, Kessel-Lo (BE); Chris Van Haesendonck, Weerde (BE)

(72) Inventors: Maarten Gysemans, Schriek (BE); Johan Snauwaert, Kessel-Lo (BE); Chris Van Haesendonck, Weerde (BE)

(73) Assignee: KATHOLIEKE UNIVERSITEIT LEUVEN, Leuven (BE)

(21) Appl. No.: 13/808,859

(22) PCT Filed: Jul. 8, 2011
(86) PCT No.: PCT/BE11/00043

§ 371 Date: Jan. 7, 2013

(30) Foreign Application Priority Data
Jul. 8, 2010 (GB) 1011503.8
Mar. 1, 2011 (GB) 1103409.7

(51) Int. Cl. A61K 31/655; A61K 38/43; A61K 38/02; A61K 39/395

(52) U.S. Cl. CPC A61K 31/655; A61K 39/395; A61K 38/43; A61K 38/02
USPC 424/400; 514/150; 424/130.1; 424/94.1; 514/2.4

(57) ABSTRACT

Methods and compositions for treating biofilms and diseases associated with amyloidosis such as Alzheimer's, Parkinson's and Huntington's disease, and Type 2 diabetes, by destroying amyloid fibrils in a two-step treatment are disclosed. The first step consists of binding to the amyloid fibril an intercalating molecule with a negatively charged group such as Congo red. The second step consists of adding metal ions, such as silver, gold(I), copper(I), palladium or lead ions or metal colloids of silver or gold, which destabilize the amyloid-dye complex. This process results in a disintegration of the fibril into peptide monomers and small aggregates of monomers.

### FIELD OF THE INVENTION

Present invention relate generally to compositions and methods for removal or destruction of amyloid fibril or amyloid adhesin comprising aggregates. It relates to methods and compositions for destabilizing or destructing amyloid fibrils and amyloid adhesin comprising amyloid aggregates and biofilms, respectively, and to methods and compositions for diseases associated with amyloidosis and diseases associated with microbial biofilms.

Targeting the structural stability of amyloid fibrils is relevant for the removal of amyloids and biofilms. An embodiment of present invention relates to the treatment of amyloidosis or amyloid-associated diseases by affecting the structural stability of amyloid deposits. Amyloidosis refers to a variety of conditions in which abnormal deposits of amyloid proteins are formed in organs and tissues. The accumulation of amyloid deposits in the brain is a hallmark for neurodegenerative diseases such as Alzheimer's disease and Parkinsons disease. The present invention provides methods and compositions to target the structural stability of these amyloid deposits.

On the other hand, amyloid adhesins are abundant in natural biofilms. The compounds, compositions and methods of present invention are also used to control microbial growth, in particular bacterial growth, and for reduction in microbial colonization. More particularly, the present invention is also directed to the field of medical treatment and prevention of infection diseases; in particular, to use of therapeutic compositions containing the biofilm formation inhibitors of present invention to reduce or eliminate colonization with potentially pathogenic bacteria (including bacterial strains resistant to many or most commonly used antimicrobial agents), thereby reducing the risk of subsequent disease occurrence.

Furthermore, the present invention relates to compounds and to compositions, devices and methods involving these compounds for inhibiting, reducing or preventing the formation of a biofilm on a surface such as a surface of pipelines, catheters, implants, teeth, urethra or lungs of a cystic fibrosis patient. These compounds, compositions, devices and methods of present invention are in particular embodiments used 1) for preventing biofilm formation in a tissue to reduce the risk of, prevent, control or treat chronic bacterial infection or sepsis, 2) for sanitation or when applied to a substrate providing protection against biofilm formation on said substrate. Their uses in methods, composition and devices for controlling and/or preventing microbial biofilm formation in natural, clinical, and industrial settings are also disclosed.

### BACKGROUND OF THE INVENTION

Amyloid aggregates occur in several disorders. Diseases featuring amyloids are for instance diseases of the group Alzheimer's disease, Diabetes mellitus type 2, Parkinson's disease, Transmissible spongiform encephalopathy e.g. Bovine spongiform encephalopathy, Huntington's disease, Medullary carcinoma of the thyroid Cardiac arrhythmias, Isolated atrial amyloidosis, Atherosclerosis, Rheumatoid arthritis, Aortic medial amyloid, Prolactinomas, Familial amyloid polyneuropathy, Hereditary non-neuropathic systemic amyloidosis, Dialysis related amyloidosis, Finnish amyloidosis, Lattice corneal dystrophy, Cerebral amyloid angiopathy, Cerebral amyloid angiopathy (Icelandic type), systemic AL amyloidosis, Sporadic Inclusion Body Myositis or infectious disorders involving biofilms. Present invention provides methods or compositions to destruct such amyloid and to treat these disorders.

Neurodegenerative diseases related to Alzheimer's disease (AD) constitute about two thirds of the dementia cases. The other neurodegenerative diseases such as Parkinson's disease and Huntington's disease make up the majority of the remaining cases. The pathological hallmarks of AD are neuronal loss, extracellular plaques containing the peptide β-amyloid, and intraneuronal tangles composed of a hyperphosphorylated form of the microtubular protein tau.

No effective treatment to delay or to halt the progression of neurodegenerative diseases is available today. No treatment has been proven to stop AD. The U.S. Food and Drug Administration has approved some pharmaceuticals to treat AD such as donepezil (Aricept®), rivastigmine (Exelon®), or galantamine (Razadyne®) and memantine (Namenda®). However, these drugs do not stop or reverse AD and appear to help patients only for months to a few years.

Thus, there is a need in the art for treating neurodegenerative diseases. The neurodegenerative disorders have a common molecular basis, i.e amyloid fibrils which are neurotoxic. The insoluble fibrils consist of intertwined protofilaments characterized by a specific arrangement of the β-sheets in the secondary structure of the proteins. The ribbon-like β-sheets are parallel to the fibril axis and consist of packed monomers connected laterally through stable hydrogen bonds between their β-strand regions which are arranged perpendicular to the protofilament axis (J. L. Jiménez et al., Proc. Natl. Acad. Sci. USA 99, 9196-9201 (2002)).

Discovery and identification of new compounds or agents as potential therapeutics to prevent or remove amyloid formation, deposition, accumulation and/or persistence that occurs in Alzheimer's disease, Parkinson's disease, type II diabetes, and other amyloidoses related diseases are desperately sought.

The present invention provides such novel method for treating neurodegenerative disorders by destroying amyloid fibrils in a two-step treatment. The first step consists of binding to the amyloid fibril an intercalating molecule with a negatively charged group such as Congo red (CR). After the intercalation process is complete and the excess Congo red is removed by natural excretion, the second step or destruction of the amyloid fibrils is initiated by adding monovalent metal ions, such as silver, gold(I) or copper(I) ions or by adding divalent ions such as palladium, lead, zinc or metal colloids of silver or gold with dimensions of a few nanometers. In this second step, the interaction between the intercalated CR in the fibril and the metal ions or colloids destabilizes the amyloid-dye complex. This process results in the disintegration of the amyloid fibrils into protein monomers. Metal colloids are metallic nanoparticles (5-25 000 atoms) with metallic ions adsorbed on their surface and surrounded by a shell of spatially distributed counter ions. The particle size controls the surface area and therefore the effectiveness of the colloidal silver or gold suspension.

Many bacteria produce functional amyloid. There is a widespread abundance of functional bacterial amyloid in mycolata and other gram-positive bacteria. Such amyloid or amyloid structures comprise aggregated fibers of insoluble protein in web-like sheets. Several infectious bacteria use amyloids to attach to host cells and to build biofilms, which are bacterial communities bound together in a film that helps resist antibiotics and immune attacks. Present invention provides methods or compositions to destruct such amyloid or to open biofilms comprising such amyloid to help, assist or enhance antibiotics and immune attacks

Next to the treatment of amyloid diseases, the disintegration of amyloid fibrils is also relevant for the destruction of biofilms. Biofilms are complex aggregates of microorganisms and are abundant in both natural and industrial environments, for instance on the inner walls of water and sewage pipes. In such systems, biofilms are a source of corrosion and clogging of the pipes. In medicine, biofilms are involved in numerous bacterial infections in the body, occur on the teeth where they form dental plaque and contribute to tooth decay, and contaminate implanted medical devices such as prostheses and catheters. A biofilm provides the bacteria included in it with an environment that allows them to cooperate and interact, and protects the bacteria from detergents and antibiotics. Because of this, biofilms are an important concern to public health.

Thus, a key aspect in the treatment of infectious diseases and oral infections, and the maintenance of industrial installations, is the effective removal of biofilms. The presence of amyloid fibrils as an important structural component of the extracellular matrix of biofilms has been demonstrated for films produced by Bacillus subtilis and curli pili in E. Coli. Recent data suggest that amyloid fibrils are widespread in biofilms (P. Larsen et al., Env. Microbiol. 9, 3077-3090 (2007)). The present invention provides a novel method to directly target the amyloid fibrils and thereby disrupt the structure of the extracellular matrix of the biofilm. This leaves the interior of the biofilm accessible and paves the way for treating the bacteria with traditional antibiotics and detergents. In this respect, the use of silver ions in the procedure is an added benefit as silver itself has an antibacterial effect.

Despite the focus of modern microbiology research on pure culture, planktonic bacteria, it is now recognized that most bacteria found in natural, clinical, and industrial settings persist in biofilms (Davey, M. E. and O'Toole, G. A. 2000. Microbiol Mol Bioly Rev. 64:847-867). Biofilms are structured communities of bacterial cells enclosed in a self-produced polymeric matrix and adherent to an inert or living surface (Costerton, J. W., Stewart, P.S., Greenberg, E. P. 1999. Bacterial biofilms: a common cause of persistent infections. Science 284:1318-1322). The matrix is formed by extracellular polymeric substances (EPS), which consists largely of polysaccharide and smaller amounts of protein and DNA. Patches of cells are interspersed in this EPS matrix which itself varies in density. This creates a heterogeneous structure with open areas where water channels are formed, allowing nutrients to enter the lower layers of the biofilm and, in addition, allowing waste products to be removed (Davey, M. E. and O'Toole, G. A. 2000. Microbiol Mol Bioly Rev. 64:847-867; Dunne, M. 2002. Clin Microbiol Rev. 15:155-166). Biofilm cells have profound changes in gene expression and cell physiology compared with planctonic cells and multiple genetic pathways mediate the regulation of the biofilm formation (G. O'Toole, H. B. Kaplan and R. Kolter. 2000. Biofilm formation as microbial development. Annu Rev Microbiol 54: 49-79; Lazazzera, B. E. 2005. Lessons from DNA microarray analysis: the gene expression profile of biofilms. Curr. Opin. Microbiol. 8(2):222-7). Microorganisms in biofilms thus form microbial colonies or condominiums that make it easy for the microorganisms to carry out chemical reactions that are impossible for a single microbe. The methods and compositions of present invention allows to destruct amyloid structures in said biofilms, open said biofilms and to decrease the biofilm persistence towards antibacterial compounds

Several documents are cited throughout the text of this specification. Each of the documents herein (including any manufacturer's specifications, instructions etc.) are hereby incorporated by reference; however, there is no admission that any document cited is indeed prior art of the present invention.

### SUMMARY OF THE INVENTION

The present invention solves the problems of the related art by disintegrating amyloid fibrils in tissues and organs, for instance the disintegration of the neurotoxic amyloid fibrils in the brain. In the context of biofilms, the amyloid fibrils constitute an important structural component of the extracellular matrix of the biofilms.

The present invention relates generally to amyloidosis linked disorders and, more particularly to a method for treating Alzheimer's, Parkinson's, and Huntington's disease, and Type 2 diabetes. These diseases are characterized by an accumulation of protein aggregates, which have very stable anti-parallel β-sheets that are the basic structure that can be found in all amyloids.

Amyloidosis includes a variety of diseases characterized by an accumulation of amyloid material in the organs or tissues of the body. This accumulation can impair vital functions. Diseases associated with amyloidosis include Alzheimer's disease (AD), Down's syndrome, progressive supranuclear palsy, multiple sclerosis, and adult-onset diabetes. Localized amyloidosis is associated with cognitive decline (senile cerebral amyloidosis; AD), heart disease (senile cardiac amyloidosis), endocrine tumors (thyroid cancer), and adult onset diabetes, diseases which are found in millions of people.

Furthermore, the disintegration of amyloid fibrils is also relevant for the removal of biofilms. A biofilm is a complex aggregate of microorganisms on a solid or liquid interface. Cells in the biofilm adhere to each other or to a surface and are embedded in a matrix of extracellular polymeric substance (EPS), which is produced by the cells themselves. The EPS matrix protects the cells comprised in it and facilitates communication among them through biochemical signals. Biofilms are abundant in both natural and industrial environments and can occur under extreme temperatures and chemical conditions such as acidity. Examples include biofilms on the bottom of rivers or the surface of stagnant pools, or in an industrial environment on the inner wall of water and sewage pipes. In such systems they are the cause of several adverse effects, such as clogging of the pipes and reduction of the efficiency of heating or cooling water systems. Attached to the metal surfaces of piping, biofilms are a source of corrosion and pose a substantial problem in marine engineering systems, such as pipelines of the offshore oil and gas industry. Examples in medicine include biofilms on the teeth of animals and humans, known as dental plaque, which contribute to tooth decay. Biofilms are also found on the surfaces of implanted devices such as prostheses or catheters. Furthermore, they are involved in numerous microbial infections in the body such as sinusitis, urinary tract infections or middle ear infections. The presence of biofilms in the body poses an important health problem as the extracellular matrix allows the bacteria to cooperate and interact in various ways. One benefit of this dense and protected environment is an increased resistance to detergents and antibiotics. The dense extracellular matrix and the outer layer of cells protects the interior of the bacterial colony and in some cases, antibiotic resistance can be increased a thousand fold (S. Stewart et al., Lancet 358, 135-138 (2001)).

In accordance with the purpose of the invention, as embodied and broadly described herein, the invention is broadly drawn to a method to destabilize or destroy amyloid fibril deposits.

One aspect of the invention is a novel method for treating neurodegenerative disorders, such as Alzheimer's, Parkinson's, and Huntington's disease, and type 2 diabetes, by disintegration of amyloid fibrils. There is no treatment available to cure or to stabilize these diseases.

Another aspect of the invention is a cure by targeting and destroying the amyloid fibril deposits that are thought to be associated with this pathology. The treatment consists of binding an intercalating molecule with a negatively charged group such as Congo red to the amyloid fibril. After the excess of Congo red is removed by natural excretion, the destruction of this amyloid-CR complex is initiated by adding monovalent metal ions, such as silver, gold(I) or copper(I) ions or by adding divalent ions such as palladium, lead or zinc or metal colloids such as silver or gold colloids. The positively charged metal ions or metal colloids interfere with the electrostatic bond between the negatively charged group of the intercalated molecule, CR, and the basic amino acid side chain of the fibril. This induces a destabilization of the amyloid-CR complex followed by a disintegration of the fibril into protein monomers and small aggregates of monomers.

Yet another aspect of the invention is the destabilizing of biofilms by targeting and destroying the amyloid fibrils that form an important structural component of the biofilm extracellular matrix. The destruction of the amyloid fibrils disrupts the structural integrity of the biofilm, making the bacteria inside vulnerable to antibiotics and detergents.

Some embodiments of the invention are set forth in claim format directly below:

An embodiment of present invention concerns a pharmaceutical composition comprising a pharmaceutically acceptable carrier and the substances (i) a sodium salt or any pharmaceutical acceptable salt of benzidinediazo-bis-1-naphthylamine-4-sulfonic acid or of a pharmaceutically acceptable derivative of benzidinediazo-bis-1-naphthylamine-4-sulfonic acid and (ii) a metal ion. It further concerns the pharmaceutical composition of this previous embodiment, such composition comprising and (i) benzidinediazo-bis-1-naphthylamine-4-sulfonic acid or a sodium salt or any pharmaceutically acceptable salt thereof and (ii) a metal ion and a pharmaceutically acceptable excipient; or such composition comprising and (i) disodium 3,3′-[[1,1′-biphenyl]-4,4′-diylbis(azo)]bis(4-aminonaphthalene-1-sulphonate) and 2) a metal ion and a pharmaceutically acceptable excipient. The benzidinediazo-bis-1-naphthylamine-4-sulfonic acid salt is in a particular embodiment Congo red. The metal ion can be a positive ion. It can for instance be silver or it can be a metal ion which is an ion of the group consisting of silver, gold(I), copper(I), palladium, lead, zinc, metal colloid of silver and metal colloid of gold.

In a particular embodiment the above described pharmaceutical composition comprising and (i) dipotassium 3,3′-[[1,1′-biphenyl]-4,4′-diylbis(azo)]bis(4-aminonaphthalene-1-sulphonate) and (ii) a metal ion and a pharmaceutically acceptable excipient. This composition can further comprise an agent that modifies the release of the substance, a glidant/diluent, a filler, a binder/disintegrant, a lubricant, a subcoat, a topcoat, an enteric coat, and any combination thereof. In this composition the substances can be present in an amount sufficient to inhibit cellular toxicity induced by amyloid, and a pharmaceutically acceptable vehicle; or the substances are present in an amount sufficient to inhibit amyloidosis induced neurodegeneration. In this composition the substances can be present in an amount sufficient to destabilize amyloid fibril deposits or the substances can be present in an amount sufficient to destroy amyloid fibril deposits. In this composition the substances can be present in an amount sufficient to treat amyloidosis and prevent death of beta-cells in type 2 diabetes mellitus.

In particular embodiments of present invention the above described compositions are for use in a treatment of an amyloid-related disease such as a cerebral amyloid angiopathy or an Alzheimer's disease. In yet another particular embodiments of present invention the above describe compositions are formulated for oral administration.

Another aspect of the present invention provides the pharmaceutical pack of present invention comprising the substances and (i) a sodium salt or any pharmaceutical acceptable salt of benzidinediazo-bis-1-naphthylamine-4-sulfonic acid or of a pharmaceutically acceptable derivative of benzidinediazo-bis-1-naphthylamine-4-sulfonic acid and (ii) a metal ion. This pharmaceutical pack can comprise (i) benzidinediazo-bis-1-naphthylamine-4-sulfonic acid or a sodium salt or any pharmaceutically acceptable salt thereof and (ii) a metal ion and a pharmaceutically acceptable excipient or this pharmaceutical pack can comprise (i) disodium 3,3′-[[1,1′-biphenyl]-4,4′-diylbis(azo)]bis(4-aminonaphthalene-1-sulphonate) and (ii) a metal ion and a pharmaceutically acceptable excipient. In a particular embodiment the benzidinediazo-bis-1-naphthylamine-4-sulfonic acid salt is Congo red. In yet another particular embodiment the metal ion is a positive ion, for instance the metal ion is silver or for instance the metal ion is an ion of the group consisting of silver, gold(I), copper(I), palladium, lead, zinc, metal colloid of silver and metal colloid of gold. n yet another particular embodiment the pharmaceutical pack comprises and (i) dipotassium 3,3′-[[1,1′-biphenyl]-4,4′-diylbis(azo)]bis(4-aminonaphthalene-1-sulphonate) and (ii) a metal ion and a pharmaceutically acceptable excipient. It can also involve in a particular embodiment a combination of the above.

Another aspect of the present invention provides the pharmaceutical pack of any one of the previous embodiments, wherein the substances (i) and (ii) are formulated separately and in individual dosage amounts; or wherein the substances (i) and (ii) are formulated together and in individual dosage amounts.

Another aspect of the present invention provides the composition or pack of any one of the previous embodiments for use in a treatment of amyloid-associated diseases; or for use in a treatment to cure or to stabilize amyloid-associated diseases; or for use in a treatment of amyloid-associated diseases wherein the amyloid-related disease or condition is treated prophylactically or therapeutically; or for use in a treatment of amyloid-associated diseases wherein the amyloid-associated disease is Alzheimer's disease.

Another aspect of the present invention provides the composition or pack of any one of the previous embodiments for use in a treatment of amyloid-associated diseases wherein the amyloid-associated diseases comprises Type 2 diabetes mellitus, amyloid A (reactive), secondary amyloidosis, familial mediterranean fever, familial amyloid nephropathy with urticaria and deafness (Muckle-wells Syndrome), amyloid lambda L-chain or amyloid kappa L-chain (idiopathic, myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosises), AIAPP or amylin insulinoma, atrial naturetic factor (isolated atrial amyloid), procalcitonin (medullary carcinoma of the thyroid), gelsolin (familial amyloidosis (Finnish)), cystatin C (hereditary cerebral hemorrhage with amyloidosis (Icelandic)), AApo-A-I (familial amyloidotic polyneuropatha-lowa), AApo-A-II (accelerated senescence in mice), fibrinogen-associated amyloid; and Asor or Pr P-27 (scrapie, Creutzfeld Jacob disease, Gertsmann-Straussler-Scheinker syndrome, bovine spongiform encephalitis) and persons who are homozygous for the apolipoprotein E4 allele; or wherein the amyloid-associated diseases comprises a disease or condition selected from the group consisting of Alzheimer's disease, cerebral amyloid angiopathy, inclusion body myositis, macular degeneration, Down's syndrome, mild cognitive impairment, cognitive decline and hereditary cerebral hemorrhage.

Another aspect of the present invention provides the composition or pack of any one of the previous embodiments for use in a treatment for disintegrating or destabilising accumulated amyloid material in the organs or tissues of the body.

Another aspect of the present invention provides the composition or pack of any one of the previous embodiments; comprising pharmaceutically effective amount of the substances for use in a treatment of amyloid-associated diseases

In one embodiment the composition or pack of any one of the previous embodiments has the negatively charged secondary diazo dye or the negatively charged benzidinediazo-bis-1-naphthylamine-4-sulfonic administered at 0.05-200 milligrams of substance. In yet another embodiment the composition or pack of any one of the previous embodiments has the negatively charged secondary diazo dye or the negatively charged benzidinediazo-bis-1-naphthylamine-4-sulfonic administered at 0.1-20 milligrams of substance.

The composition or pack of any one of the previous embodiments can be designed to have the metal cation administered in a dosage amount equivalent to 0.05-1000 milligrams of negatively charged secondary diazo dye or the negatively charged benzidinediazo-bis-1-naphthylamine-4-sulfonic or to have metal cation administered in a dosage amount equivalent to 0.1-500 milligrams of negatively charged secondary diazo dye or the negatively charged benzidinediazo-bis-1-naphthylamine-4-sulfonic.

Another aspect of the present invention provides the composition or pack of any one of the previous embodiments; comprising pharmaceutically effective amount of the substances for use in a treatment of amyloid-associated diseases wherein the amyloid-related disease is a disease associated with formation of a biofilm in a subject or patient in need of such treatment; or wherein the amyloid-related disease is a disease associated with formation of a biofilm with amyloid fibrils in a subject or patient in need of such treatment; or wherein the amyloid-related disease is a microbial infection or a sepsis, or wherein the amyloid-related disease is a condition associated with a microbial infection or for decreasing bacterial growth in an animal subject or a human patient in need of such treatment.

Another aspect of the present invention provides the composition or pack of any one of the previous embodiments; comprising pharmaceutically effective amount of the substances for use in a treatment of amyloid-associated diseases, wherein the amyloid-related disease is a disease associated with formation of a biofilm in a subject or patient in need of such treatment; or wherein the amyloid-related disease is a condition associated with an infection in a subject or patient in need of such treatment a composition; or wherein the amyloid-related disease is for increasing susceptibility to cytotoxic effects of antibacterial agents; or wherein the amyloid-related disease wherein the patient has a wound selected from the group consisting of an ulcer, a laceration, a deep penetrating wound and a surgical wound.

Another aspect of the present invention provides the composition or pack of any one of the previous embodiments for use in a treatment for suppressing microbial biofilm growth wherever said suppression is desired or for suppressing microbial biofilm formation and decreasing bacterial growth wherever said suppression is desired.

One embodiment concerns the composition or pack of any one of the previous embodiments, wherein the composition or pack is selected from the group consisting of an oral tablet, capsule or liquid, a nasal aerosol, a throat wash, a mouth wash or gargle, a toothpaste, and a topical ointment.

Yet another embodiment concerns the composition or pack of any one of the previous embodiments, wherein the composition or pack is selected from the group consisting of tampons, rinses, creams, and aerosols.

Yet another embodiment concerns the composition or pack of any one of the previous embodiments, wherein the composition is selected from the group consisting of soap, hair shampoo, toothbrushes, tooth paste, cotton swabs, antiperspirant, facial tissue, mouthwash, nail files, skin cleansers and toilet paper.

Yet another embodiment concerns the composition or pack of any one of the previous embodiments, wherein the composition is a topical ointment, an irrigation solution or a component of a wound dressing.

Yet another embodiment concerns the composition or pack of any one of the previous embodiments for use in a treatment by administering intravesicularly, topically, orally, rectally, ocularly, otically, nasally, parenterally, vaginally, intravenously, directly into an infected site, directly onto an indwelling prosthetic device or catheter.

Yet another embodiment concerns the composition or pack of any one of the previous embodiments for reducing the risk of bacterial infection or sepsis in a person colonized with pathogenic bacteria, wherein said treatment occurs prior to said colonized person developing an illness due to said pathogenic bacteria.

Yet another embodiment concerns the composition or pack of any one of the previous embodiments, for use in a treatment for reducing the risk of bacterial infection or sepsis in a person colonized with pathogenic bacteria, wherein said treatment reduces the risk of bacterial infection or sepsis in said colonized person.

Yet another embodiment concerns the composition or pack of any one of the previous embodiments, for use in a treatment for reducing the risk of bacterial infection or sepsis in a person, wherein said person is an immunocompromised patient selected from the group consisting of leukemia patients, lymphoma patients, carcinoma patients, sarcoma patients, allogeneic transplant patients, congenital or acquired immunodeficiency patients, cystic fibrosis patients, and AIDS patients.

Yet another embodiment concerns the composition or pack of any one of the previous embodiments, for use in a treatment of inhibiting or preventing the formation of a biofilm or condition associated with formation of a biofilm on a biotic surface or in a biotic substrate.

Yet another embodiment concerns the composition or pack of any one of the previous embodiments, wherein the amyloid-related disease is a disease associated with formation of a biofilm in a subject or patient in need of such treatment and wherein the biofilm comprises more than one species of bacteria.

Yet another embodiment concerns the composition or pack of any one of the previous embodiments, wherein the amyloid-related disease is a disease associated with formation of a biofilm in a subject or patient in need of such treatment and wherein the biofilm comprises gram negative bacteria.

Yet another embodiment concerns the composition or pack of any one of the previous embodiments, wherein the amyloid-related disease is a disease associated with formation of a biofilm in a subject or patient in need of such treatment and wherein the biofilm further comprises gram positive bacteria; or wherein the amyloid-related disease is a disease associated with formation of a biofilm in a subject or patient in need of such treatment and wherein the biotic surface or the biotic substrate is associated with bacterial infection; or wherein the amyloid-related disease is a disease associated with formation of a biofilm in a subject or patient in need of such treatment and wherein the biotic surface or the biotic substrate is an epithelial or a mucosal layer; or wherein the amyloid-related disease is a disease associated with formation of a biofilm in a subject or patient in need of such treatment and wherein the surface is an epithelial or mucosal surface of a mammal; or the composition or pack of embodiment 56, wherein the amyloid-related disease is a disease associated with formation of a biofilm in a subject or patient in need of such treatment and wherein the biotic surface or the biotic substrate is a mucosa selected from the group consisting of mouth, vagina, astrointestinal tract and oesophageal tract, or wherein the amyloid-related disease is a disease associated with formation of a biofilm in a subject or patient in need of such treatment and wherein the surface is the surface of a tooth, or, wherein the amyloid-related disease is a disease associated with formation of a biofilm in a subject or patient in need of such treatment and wherein the condition is an oral infection.

Yet another embodiment concerns the composition or pack of any one of the previous embodiments, wherein the substance is to be co-administered with one or more antibacterial agents; or wherein the substance is to be co-administered with one or more antibacterial agents selected from the group consisting of antibiotics, antibodies, antibacterial enzymes, peptides, lantibiotics, lanthione-containing molecules and bacteriophages; or wherein the substances are to be added in a single dose, in multiple doses, in multiple doses that are added on separate days, in multiple doses that are added on the same day, or are added continuously; or wherein the microbial organism or microbe is a bacterium.

Yet another embodiment concerns the composition or pack of any one of the previous embodiments for use in a treatment to reduce biofilm resistance to chemical (for instance antimicrobial) and mechanical treatments.

Some embodiments of the invention are set forth in claim format directly below:

1. A composition for preventing, suppressing or removing a microbial biofilm or amyloid adhesions comprising biofilms, characterized in that it comprises the substances (i) a sodium salt or any pharmaceutically acceptable salt of benzidinediazo-bis-1-naphthylamine-4-sulfonic acid or of a pharmaceutically acceptable derivative of benzidinediazo-bis-1-naphthylamine-4-sulfonic acid and (ii) a metal ion.
2. The composition according to embodiment 1, comprising and (i) benzidinediazo-bis-1-naphthylamine-4-sulfonic acid or a sodium salt or any pharmaceutically acceptable salt thereof and (ii) a metal ion and a pharmaceutically acceptable excipient.
3. The composition according to embodiment 1, comprising and (i) disodium 3,3′-[[1,1′-biphenyl]-4,4′-diylbis(azo)]bis(4-aminonaphthalene-1-sulphonate) and 2) a metal ion and a pharmaceutically acceptable excipient.
4. The composition according to embodiment 1, wherein the benzidinediazo-bis-1-naphthylamine-4-sulfonic acid salt is Congo red.
5. The composition according to embodiment 1, wherein the metal ion is a positive ion.
6. The composition according to embodiment 1, wherein the metal ion is silver.
7. The composition according to embodiment 1, wherein metal ion is an ion of the group consisting of silver, gold(I), copper(I), palladium, lead, zinc, metal colloid of silver and metal colloid of gold.
8. The composition according to embodiment 1, comprising and (i) dipotassium 3,3′-[[1,1′-biphenyl]-4,4′-diylbis(azo)]bis(4-aminonaphthalene-1-sulphonate) and (ii) a metal ion and a pharmaceutically acceptable excipient.
9. The composition of any one of the previous embodiments, that further comprises an agent that modifies the release of the substance, a glidant/diluent, a filler, a binder/disintegrant, a lubricant, a subcoat, a topcoat, an enteric coat, and any combination thereof.
10. The composition according to embodiment 1, wherein the substances are present in an amount sufficient to destabilize amyloid adhesion deposits in a biofilm.
11. The composition according to embodiment 1, wherein the substances are present in an amount sufficient to destroy amyloid adhesion deposits in a biofilm.
12. A pack comprising the substances and (i) a sodium salt or any acceptable salt of benzidinediazo-bis-1-naphthylamine-4-sulfonic acid or of an acceptable derivative of benzidinediazo-bis-1-naphthylamine-4-sulfonic acid and (ii) a metal ion.
13. The pack according to embodiment 12, comprising and (i) benzidinediazo-bis-1-naphthylamine-4-sulfonic acid or a sodium salt or any pharmaceutically acceptable salt thereof and (ii) a metal ion and a pharmaceutically acceptable excipient.
14. The pack according to embodiment 12, comprising and (i) disodium 3,3′-[[1,1′-biphenyl]-4,4′-diylbis(azo)]bis(4-aminonaphthalene-1-sulphonate) and (ii) a metal ion and a pharmaceutically acceptable excipient.
15. The pack according to embodiment 12 wherein the benzidinediazo-bis-1-naphthylamine-4-sulfonic acid salt is Congo red.
16. The pack according to embodiment 12, wherein the metal ion is a positive ion.
17. The pack according to embodiment 12, wherein the metal ion is silver.
18. The pack according to embodiment 12, wherein metal ion is an ion of the group consisting of silver, gold(I), copper(I), palladium, lead, zinc, metal colloid of silver and metal colloid of gold.
19. The pack according to embodiment 12, comprising (i) dipotassium 3,3′-[[1,1′-biphenyl]-4,4′-diylbis(azo)]bis(4-aminonaphthalene-1-sulphonate) and (ii) a metal ion and a pharmaceutically acceptable excipient.
20. The pack of any one of the previous embodiments 12 to 19, wherein the substances (i) and (ii) are formulated separately and in individual dosage amounts.
21. The pack of any one of the previous embodiments 12 to 19, wherein the substances (i) and (ii) are formulated together and in individual dosage amounts.
22. The composition or pack of any one of the previous embodiments 1 to 21; wherein said the negatively charged secondary diazo dye or the negatively charged benzidinediazo-bis-1-naphthylamine-4-sulfonic is administered at 0.05-200 milligrams of substance.
23. The composition or pack of any one of the previous embodiments 1 to 21; wherein said the negatively charged secondary diazo dye or the negatively charged benzidinediazo-bis-1-naphthylamine-4-sulfonic is administered at 0.1-20 milligrams of substance.
24. The composition or pack of any one of the previous embodiments 1 to 21; wherein said metal cation is administered in a dosage amount equivalent to 0.05-1000 milligrams of negatively charged secondary diazo dye or the negatively charged benzidinediazo-bis-1-naphthylamine-4-sulfonic.
25. The composition or pack of any one of the previous embodiments 1 to 21; wherein said metal cation is administered in a dosage amount equivalent to 0.1-500 milligrams of negatively charged secondary diazo dye or the negatively charged benzidinediazo-bis-1-naphthylamine-4-sulfonic.
26. The composition or pack according to any one of the embodiments 1 to 25, wherein the substances are to be co-administered with one or more antibacterial agents.
27. The composition or pack according to any one of the embodiments 1 to 25, wherein the substance is to be co-administered with one or more antibacterial agents selected from the group consisting of antibiotics, antibodies, antibacterial enzymes, peptides, lantibiotics, lanthione-containing molecules and bacteriophages;
28. The composition or pack according to any one of the embodiments 1 to 25, wherein the substances are to be added in a single dose, in multiple doses, in multiple doses that are added on separate days, in multiple doses that are added on the same day, or are added continuously.
29. The composition or pack according to any one of the embodiments 1 to 25, wherein the microbial organism or microbe is a bacterium.
30. The composition or pack according to any of the previous embodiments further comprising a detergent.
31. The composition or pack according to any of the previous embodiments, wherein the any of the two substances is in a controlled-release formulation, or is contained in a device that permits controlled release of the compound.
32. The use of the composition or pack according to any of the previous embodiments 1 to 31, for destroying or destabilising natural biofilms.
33. The use of the composition or pack according to any of the previous embodiments 1 to 31, preventing, suppressing or removing a microbial biofilm or amyloid adhesions comprising biofilms.
34. The use of the pack, composition or method according to any of the previous embodiments 1 to 18 for the prevention, suppression or reduction of biofilm formation on an abiotic surface, or to prevent, suppress or reduce viable microbial growth on an abiotic surface; or to prevent or suppress microbial contamination on an abiotic surface.
35. Use according to any one of the embodiments 32 to 34, wherein the abiotic surface is a medical device, fluid storage apparatus, fluid delivery apparatus, surface of a food processing facility or surgical implement.
36. Use according to any one of the embodiments 32 to 34, wherein the medical device is a surgical implant.
37. Use according to any one of the embodiments 32 to 34, wherein the abiotic surface is, or forms part of, a contact lens.
38. Use according to any one of the embodiments 32 to 34, wherein the abiotic surface is a filter or a water delivery pipe.
39. Use according to any one of the embodiments 32 to 34, wherein the abiotic surface is impregnated or coated with the compound.
40. Use according to any one of the embodiments 32 to 34, wherein the abiotic surface is metal, plastic, ceramic, polystyrene or glass.
41. Use according to any one of the embodiments 32 to 34, wherein the biofilm comprises more than one species of bacteria.
42. Use according to any one of the embodiments 32 to 34, wherein the biofilm comprises gram negative bacteria.
43. Use according to any one of the embodiments 32 to 34, for treating a surgical implement prior to surgery.
44. A non living object or an abiotic surface protected against biofilm formation said object or surface to which composition or pack according to any of the previous embodiments 1 to 31 is applied on and/or incorporated or and/or coated on in an amount effective to reduce or inhibit biofilm formation.
45. The object according to embodiment 44, wherein said object has a solid surface.
46. The object according to embodiment 44, wherein the object is or the surface is of a medical facility that has to be prevented of being colonized by microbial.
47. The object according to embodiment 44, wherein the object is or the surface is of a concrete that has to be prevented of being colonized by microbial.
48. The object according to embodiment 44, wherein the object is or the surface is of packaging material for packing foodstuff for instance foodstuff selected from the group consisting of: produce, cut fruits and cut vegetables.
49. The object according to embodiment 44, wherein the object is a fabric.
50. The fabric according to embodiment 49, wherein the fabric is a woven material.
51. The fabric according to embodiment 49, wherein the fabric is swab impregnated with a compound of combination thereof according to any of the previous embodiments.
52. The object according to embodiment 44, wherein the object is a medical device.
53. The medical device according to embodiment 52, whereby the medical device is a surgical implant.
54. The medical device according to embodiment 52, whereby the medical device is a contact lens.
55. The medical device according to embodiment 52, whereby the medical device is a is a dental implant, catheter, stent, guide wire or orthopaedic prosthetic.
56. The object according to embodiment 44, wherein the object is a filter or water delivery pipe.
57. The object according to embodiment 44, wherein the object is a fluid storage or fluid delivery device.
58. A paint comprising composition according to any of the previous embodiments 1 to 11 to protect a non living object or an abiotic surface protected against biofilm formation on said object or on surface whereon the paint is applied on.
59. A method of preventing microbial contamination, comprising the step of: applying to a product on which it is desired an effective amount of the composition or pack according to any of the previous embodiments 1 to 31.
60. The method according to embodiment 59, for preventing spoilage, comprising the step of: applying to a product on which it is desired to prevent spoilage an effective amount of a compound or combinations thereaccording to any of the previous embodiments:
61. The method according to embodiment 59, whereby the solution is further comprised with a detergent for adding it to the product.
62. The method according to embodiment 59, for preventing, suppressing or removing a microbial biofilm of a product which is an abiotic surface, characterized in that it comprises at least the following steps, carried out simultaneously or consecutively: a solution comprising a compound or combinations thereof according to any of the previous embodiments is prepared; and said solutions are applied, by contacting it or applying to the abiotic surface to be treated.
63. The method according to embodiment 59, for imparting microbial control properties on an abiotic surface or in a abiotic substrate comprising adding or administering an effective amount of a compound or combinations of compounds of any one of the preceding embodiments.
64. The method according to embodiment 59, for imparting microbial biofilm control properties to a fluid, comprising adding a compound or combinations of compounds according to any one of the preceding embodiments to said composition.

Particular and preferred aspects of the invention are set out in the accompanying independent and dependent claims. Features from the dependent claims may be combined with features of the independent claims and with features of other dependent claims as appropriate and not merely as explicitly set out in the claims.

Thus, the claims following the detailed description are hereby expressly incorporated into this detailed description, with each claim standing on its own as a separate embodiment of this invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### DETAILED DESCRIPTION

### Detailed Description of Embodiments of the Invention

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

Other embodiments of the invention will be apparent to those skilled in the art from consideration of the specification and practice of the invention disclosed herein.

It is intended that the specification and examples be considered as exemplary only.

Each and every claim is incorporated into the specification as an embodiment of the present invention. Thus, the claims are part of the description and are a further description and are in addition to the preferred embodiments of the present invention.

Each of the claims set out a particular embodiment of the invention.

Congo red is the sodium salt of benzidinediazo-bis-1-naphthylamine-4-sulfonic acid (formula: C₃₂H₂₂N₆Na₂O₆S₂; molecular weight: 696.66 g/mol). It is a secondary diazo dye. Congo red is water soluble, yielding a red colloidal solution; its solubility is better in organic solvents such as ethanol.

A variety of human diseases demonstrate amyloid deposition and usually involve systemic organs (i.e. organs or tissues lying outside the central nervous system), with the amyloid accumulation leading to organ dysfunction or failure. These amyloid diseases (discussed below) leading to marked amyloid accumulation in a number of different organs and tissues, are known as systemic amyloidosis. In other amyloid diseases, single organs may be affected such as the pancreas in 90% of patients with type 2 diabetes. In this type of amyloid disease, the beta-cells in the islets of Langerhans in pancreas are believed to be destroyed by the accumulation of fibrillar amyloid deposits consisting primarily of a protein known as islet amyloid polypeptide (IAPP). Inhibiting or reducing such IAPP amyloid fibril formation, deposition, accumulation and persistence is believed to lead to new effective treatments for type 2 diabetes. In Alzheimer's disease, Parkinson's and “systemic” amyloid diseases, there is currently no cure or effective treatment, and the patient usually dies within 3 to 10 years from disease onset.

The amyloid diseases (amyloidoses) are classified according to the type of amyloid protein present as well as the underlying disease. Amyloid diseases have a number of common characteristics including each amyloid comprising a unique type of amyloid protein. The amyloid diseases include, but are not limited to, the amyloid associated with Alzheimer's disease, Down's syndrome, hereditary cerebral hemorrhage with amyloidosis of the Dutch type, dementia pugilistica, inclusion body myositosis (Askanas et al, Ann. Neurol. 43:521-560, 1993) and mild cognitive impairment (where the specific amyloid is referred to as beta-amyloid protein or Aβ), the amyloid associated with chronic inflammation, various forms of malignancy and familial Mediterranean fever (where the specific amyloid is referred to as AA amyloid or inflammation-associated amyloidosis), the amyloid associated with multiple myeloma and other B-cell dyscrasias (where the specific amyloid is referred to as AL amyloid), the amyloid associated with type 2 diabetes (where the specific amyloid protein is referred to as amylin or islet amyloid polypeptide or IAPP), the amyloid associated with the prion diseases including Creutzfeldt-Jakob disease, Gerstmann-Straussler syndrome, kuru and animal scrapie (where the specific amyloid is referred to as PrP amyloid), the amyloid associated with long-term hemodialysis and carpal tunnel syndrome (where the specific amyloid is referred to as α2-microglobulin amyloid), the amyloid associated with senile cardiac amyloidosis and familial amyloidotic polyneuropathy (where the specific amyloid is referred to as transthyretin or prealbumin), and the amyloid associated with endocrine tumors such as medullary carcinoma of the thyroid (where the specific amyloid is referred to as variants of procalcitonin). In addition, the α-synuclein protein which forms amyloid-like fibrils, and is Congo red and thioflavin S positive (specific stains used to detect amyloid fibrillar deposits), is found as part of Lewy bodies in the brains of patients with Parkinson's disease, Lewy body disease (Lewy in Handbuch der Neurologie, M. Lewandowski, ed., Springer, Berlin pp. 920-933, 1912; Pollanen et al, J. Neuropath. Exp. Neurol. 52:183-191, 1993; Spillantini et al, Proc. Natl. Acad. Sci. USA 95:6469-6473, 1998; Arai et al, Neurosci. Lett. 259:83-86, 1999), multiple system atrophy (Wakabayashi et al, Acta Neuropath. 96:445-452, 1998), dementia with Lewy bodies, and the Lewy body variant of Alzheimer's disease. For purposes of this disclosure, Parkinson's disease, due to the fact that fibrils develop in the brains of patients with this disease (which are Congo red and Thioflavin S positive, and which contain predominant beta-pleated sheet secondary structure), is now regarded as a disease that also displays the characteristics of an amyloid-like disease.

Systemic amyloidoses which include the amyloid associated with chronic inflammation, various forms of malignancy and familial Mediterranean fever (i.e. AA amyloid or inflammation-associated amyloidosis) (Benson and Cohen, Arth. Rheum. 22:36-42, 1979; Kamei et al, Acta Path. Jpn. 32:123-133, 1982; McAdam et al., Lancet 2:572-573, 1975; Metaxas, Kidney Int. 20:676-685, 1981), and the amyloid associated with multiple myeloma and other B-cell dyscrasias (i.e. AL amyloid) (Harada et al., J. Histochem. Cytochem. 19:1-15, 1971), as examples, are known to involve amyloid deposition in a variety of different organs and tissues generally lying outside the central nervous system. Amyloid deposition in these diseases may occur, for example, in liver, heart, spleen, gastrointestinal tract, kidney, skin, and/or lungs (Johnson et al, N. Engl. J. Med. 321:513-518, 1989). For most of these amyloidoses, there is no apparent cure or effective treatment and the consequences of amyloid deposition can be detrimental to the patient. For example, amyloid deposition in the kidney may lead to renal failure, whereas amyloid deposition in the heart may lead to heart failure. For these patients, amyloid accumulation in systemic organs leads to eventual death generally within 3-5 years. Other amyloidoses may affect a single organ or tissue such as observed with the Aβ amyloid deposits found in the brains of patients with Alzheimer's disease and Down's syndrome: the PrP amyloid deposits found in the brains of patients with Creutzfeldt-Jakob disease, Gerstmann-Straussler syndrome, and kuru; the islet amyloid (IAPP) deposits found in the islets of Langerhans in the pancreas of 90% of patients with type 2 diabetes (Johnson et al, N. Engl. J. Med. 321:513-518, 1989; Lab. Invest. 66:522 535, 1992); the α2-microglobulin amyloid deposits in the medial nerve leading to carpal tunnel syndrome as observed in patients undergoing long-term hemodialysis (Geyjo et al, Biochem. Biophys. Res. Comm. 129:701-706, 1985; Kidney Int. 30:385-390, 1986); the prealbumin/transthyretin amyloid observed in the hearts of patients with senile cardiac amyloid; and the prealbumin/transthyretin amyloid observed in peripheral nerves of patients who have familial amyloidotic polyneuropathy (Skinner and Cohen, Biochem. Biophys. Res. Comm. 99:1326-1332, 1981; Saraiva et al, J. Lab. Clin. Med. 102:590-603, 1983; J. Clin. Invest. 74:104-119, 1984; Tawara et al, J. Lab. Clin. Med. 98:811-822, 1989).

Alzheimer's disease (AD) is a progressive neurologic disease that results in the irreversible loss of neurons, particularly in the cortex and hippocampus. The clinical hallmarks are progressive impairment in memory and cognitive processes that significantly diminishes a person's functioning. It is the most prevalent form of dementia for which effective therapies are desperately needed. Alzheimer's disease results in an accumulation of extracellular plaques and intraneuronal tangles in the brain. The accumulations are composed of abnormal protein filaments referred to as amyloid fibrils. Alzheimer's disease also puts a heavy economic burden on society. A recent study estimated that the cost of caring for one Alzheimer's disease patient with severe cognitive impairments at home or in a nursing home, is more than $47,000 per year (A Guide to Understanding Alzheimer's Disease and Related Disorders). For a disease that can span from 2 to 20 years, the overall cost of Alzheimer's disease to families and to society is staggering. The annual economic toll of Alzheimer's disease in the United States in terms of health care expenses and lost wages of both patients and their caregivers is estimated at $80 to $100 billion (2003 Progress Report on Alzheimer's Disease).

Amyloid as a therapeutic target for Alzheimer's disease: Alzheimer's disease is characterized by the deposition and accumulation of a 39-43 amino acid peptide termed the beta-amyloid protein, Aβ or β/A4 (Glenner and Wong, Biochem. Biophys. Res. Comm. 120:885-890, 1984; Masters et al., Proc. Natl. Acad. Sci. USA 82:4245-4249, 1985; Husby et al., Bull. WHO 71:105-108, 1993). AP is derived by protease cleavage from larger precursor proteins termed β-amyloid precursor proteins (APPs) of which there are several alternatively spliced variants. The most abundant forms of the APPs include proteins consisting of 695, 751 and 770 amino acids (Tanzi et al., Nature 31:528-530, 1988). The small Aβ peptide is a major component that makes up the amyloid deposits of “plaques” in the brains of patients with Alzheimer's disease. In addition, Alzheimer's disease is characterized by the presence of numerous neurofibrillary “tangles”, consisting of paired helical filaments which abnormally accumulate in the neuronal cytoplasm (Grundke-Iqbal et al., Proc. Natl. Acad. Sci. USA 83:4913-4917, 1986; Kosik et al., Proc. Natl. Acad. Sci. USA 83:4044-4048, 1986; Lee et al., Science 251:675-678, 1991). The pathological hallmark of Alzheimer's disease is therefore the presence of “plaques” with amyloid being deposited in the central core of the plaques, and intracellular “tangles” consisting of or comprising amyloid fibrils. The other major type of lesion found in the Alzheimer's disease brain is the accumulation of amyloid in the walls of blood vessels, both within the brain parenchyma and in the walls of meningeal vessels that lie outside the brain. The amyloid deposits localized to the walls of blood vessels are referred to as cerebrovascular amyloid or congophilic angiopathy (Mandybur, J. Neuropath. Exp. Neurol. 45:79-90, 1986; Pardridge et al., J. Neurochem. 49:1394-1401, 1987).

For many years there has been an ongoing scientific debate as to the importance of “amyloid” in Alzheimer's disease, and whether the “plaques” and “tangles” characteristic of this disease were a cause or merely a consequence of the disease. Studies indicate that amyloid is indeed a causative factor for Alzheimer's disease and should not be regarded as merely an innocent bystander. The Alzheimer's Aβ protein in cell culture has been shown to cause degeneration of nerve cells within short periods of time (Pike et al., Br. Res. 563:311-314, 1991; J. Neurochem. 64:253-265, 1995). Studies suggest that it is the fibrillar structure (consisting of or comprising a predominant β-pleated sheet secondary structure), characteristic of all amyloids, that is responsible for the neurotoxic effects. Aβ has also been found to be neurotoxic in slice cultures of hippocampus (Harrigan et al., Neurobiol. Aging 16:779-789, 1995) and induces nerve cell death in transgenic mice (Games et al., Nature 373:523-527, 1995; Hsiao et al., Science 274:99-102, 1996). Injection of the Alzheimer's Aβ into rat brain also causes memory impairment and neuronal dysfunction (Flood et al., Proc. Natl. Acad. Sci. USA 88:3363-3366, 1991; Br. Res. 663:271-276, 1994). Probably, the most convincing evidence that Aβ amyloid is directly involved in the pathogenesis of Alzheimer's disease comes from genetic studies. It was discovered that the production of Aβ can result from mutations in the gene encoding for its precursor, β-amyloid precursor protein (Van Broeckhoven et al., Science 248:1120-1122, 1990; Murrell et al., Science 254:97-99, 1991; Haass et al., Nature Med. 1:1291-1296, 1995). The identification of mutations in the beta-amyloid precursor protein gene that cause early onset familial Alzheimer's disease is the strongest argument that amyloid is central to the pathogenetic process underlying this disease. Four reported disease-causing mutations have been discovered which demonstrate the importance of Aβ in causing familial Alzheimer's disease (reviewed in Hardy, Nature Genet. 1:233-234, 1992). All of these studies suggest that providing a drug to reduce, eliminate or prevent fibrillar Aβ formation, deposition, accumulation and/or persistence in the brains of human patients will serve as an effective therapeutic.

Parkinson's disease (PD) is a neurodegenerative movement disorder, characterized by muscle rigidity, tremor, bradykinesia and postural instability. The disease is caused by an abnormal accumulation of the protein alpha-synuclein bound to ubiquitin in brain cells, which forms proteinaceous cytoplasmic inclusions called Lewy bodies.

Huntington's disease (HD) is a progressive neurodegenerative genetic disorder which affects muscle coordination, characterized by involuntary movements called chorea. The disease affects also some cognitive functions resulting in psychiatric manifestations.

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of or comprising α-synuclein (Spillantini et al., Proc. Natl. Acad. Sci. USA 95:6469-6473, 1998; Arai et al., Neurosci. Lett. 259:83-86, 1999), an 140-amino acid protein (Ueda et al., Proc. Natl. Acad. Sci. USA 90:11282-11286, 1993). Two dominant mutations in α-synuclein causing familial early onset Parkinson's disease have been described suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998). In vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999). Most importantly, both Parkinson's disease-linked α-synuclein mutations accelerate this aggregation process, demonstrating that such in vitro studies may have relevance for Parkinson's disease pathogenesis. Alpha-synuclein aggregation and fibril formation fulfils the criteria of a nucleation-dependent polymerization process (Wood et al., J. Biol. Chem. 274:19509-19512, 1999). In this regard α-synuclein fibril formation resembles that of Alzheimer's β-amyloid protein (A13) fibrils. Alpha-synuclein recombinant protein, and non-Aβ component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and thioflavin S (demonstrating positive fluorescence) (Hashimoto et al., Brain Res. 799:301-306, 1998; Ueda et al., Proc. Natl. Acad. Sci. USA 90:11282-11286, 1993).

Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23: 457-460, 2002). The role of synucleins (and in particular, alpha-synuclein) in the etiology of a number of neurodegenerative and/or amyloid diseases has developed from several observations. Pathologically, synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form amyloid-like fibrils that recapitulated the ultrastructural features of alpha-synuclein isolated from patients with dementia with Lewy bodies, Parkinson's disease and multiple system atrophy. Additionally, the identification of mutations within the synuclein gene, albeit in rare cases of familial Parkinson's disease, demonstrated an unequivocal link between synuclein pathology and neurodegenerative diseases. The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies”.

Parkinson's disease α-synuclein fibrils, like the Aβ fibrils of Alzheimer's disease, also consist of a predominantly β-pleated sheet structure. Therefore, compounds found to inhibit Alzheimer's disease Aβ amyloid fibril formation are also anticipated to be effective in the inhibition of α-synuclein/NAC fibril formation, as shown from examples in the present invention. These compounds would therefore also serve as therapeutics for Parkinson's disease and other synucleinopathies, in addition to having efficacy as a therapeutic for Alzheimer's disease, type 2 diabetes, and other amyloid disorders.

Type 2 diabetes is a disorder characterized by high blood glucose. The present invention discloses compounds and use thereof for the preparation of a medicament to treat patients with type 2 diabetes mellitus (T2DM). The administration of these compounds results in inhibition of amyloidosis and prevention of death of pancreatic β-cells. Islet cell amyloidosis (IA) is a basic characteristic of the pathology T2DM that is associated with the death of pancreatic β-cells (Kahn et al. Diabetes 1999, 48:241-53; Hopener et al. Mol. Cell. Endocrinol. 2002, 197:205-212; O'Brien, Mol Cell Endocrinol. 2002, 197:213-219). As a consequence, β-cell mediated insulin secretion is reduced, aggravating the hyperglycemic diabetic state. Drugs currently available on the market do not prevent IA. A study in the UK analyzed the effects (11 year follow-up) of oral glycemic control agents on β-cell function and concluded that IA deposition is not diminished, and may possibly even be aggravated, and that patient β-cell function deteriorates irrespective of treatment (Tumer, Diabetes Care 1998, 21:C35-C38). Thus there is a clear need for new anti-IA therapy. The deposition of islet amyloid IA within β-cells of the pancreas is one of the main characteristics of T2DM pathology with an incidence of up to 96% (Westermark, Int J Exp Clin Invest. 1994, 1:47-60). IA has been described in humans, non-human primates and cats, but is not found in rat, mouse, rabbit, hamster, hare or dog (Hopener et al. 2002, supra). In 1987 the structure of the main component of IA was determined independently by Westermark and Cooper and designated islet amyloid polypeptide (IAPP) or amylin (Cooper et al. Proc Natl Acad. Sci. 1987, 84:8628-8632; Westermark et al. Proc Natl Acad. Sci. 1987, 84:3881-3885). It is believed that IAPP, along with insulin and glucagon, is an active islet hormone involved in the metabolic control of glucose metabolism. IAPP is co-secreted with insulin from β-cells of the pancreas. Transformation of IAPP monomers from the alpha-helix (α-helix) to beta-sheet (β-sheet) conformation results in the formation of toxic IAPP fibrils, death of β-cells and subsequent accumulation of IA. Thus the transformation of IAPP impairs insulin release and aggravates the pathology of diabetes.

Hereditary Systemic Amyloidoses:

There are many forms of hereditary systemic amyloidosis. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins. Table 1 summarizes the fibril composition of exemplary forms of these disorders.

| Fibril Peptide/Protein | Genetic Variant | Clinical Syndrome |
|---|---|---|
| ATTR protein from Transthyretin | Met30, many others | Familial amyloid polyneuropathy |
| and fragments | | (FAP), (Mainly peripheral nerves) |
| ATTR protein from Transthyretin | Thr45, Ala60, Ser84, | Cardiac involvement predominant |
| and fragments | Met111, Ile122 | without neuropathy, familial amyloid |
| | | polyneuropathy, senile systemic |
| | | amyloidosis, Tenosynovium |
| N-terminal fragment of | Arg26 | Familial amyloid polyneuropathy |
| Apolipoprotein A1 (apoAI) | | (FAP), (mainly peripheral nerves) |
| N-terminal fragment of | Arg26, Arg50, Arg 60, | Ostertag-type, non-neuropathic |
| Apoliproprotein A1 (AapoAI) | others | (predominantly visceral involvement) |
| AapoAII from Apolipoprotein | | Familial amyloidosis |
| AII | | |
| Lysozyme (Alys) | Thr56, His67 | Ostertag-type, non-neuropathic |
| | | (predominantly visceral involvement) |
| Fibrogen alpha chain fragment | Leu554, Val 526 | Cranial neuropathy with lattice corneal |
| | | dystrophy |
| Gelsolin fragment (Agel) | Asn187, Tyr187 | Cranial neuropathy with lattic corneal |
| | | dystrophy |
| Cystatin C fragment (ACys) | Glu68 | Hereditary cerebral hemorrhage |
| | | (cerebral |
| | | amyloid angiopathy)-Icelandic |
| | | type |
| β-amyloid protein (Aβ) derived | Gln693 | Hereditary cerebral hemorrhage |
| from Amyloid Precursor Protein | | (cerebral |
| (APP) | | amyloid angiopathy)-Dutch |
| | | type |
| β-amyloid protein (Aβ) derived | Ile717, Phe717, Gly717 | Familial Alzheimer's Disease |
| from Amyloid Precursor Protein | | |
| (APP) | | |
| β-amyloid protein (Aβ) derived | Gln 618 | Alzheimer's disease, Down's |
| from Amyloid Precursor Protein | | syndrome, hereditary cerebral |
| (APP), e.g., bPP 695 | | hemorrhage with amyloidosis, Dutch |
| | | type |
| β-amyloid protein (Aβ) derived | Asn670, Leu 671 | Familial Dementia-probably |
| from Amyloid Precursor Protein | | Alzheimer's Disease |
| (APP) | | |
| Prion Protein (PrP, APrPSC) | Leu102, | Familial Creutzfeldt-Jakob disease; |
| derived f om Prp precursor | Val167, | Gerstmann-Straeussler- |
| protein (51-91 insert) | Asn178, Lys200 | Scheinker syndrome |
| | | (hereditary spongiform |
| | | encephalopathies, prion |
| | | diseases) |
| AA derived from Serum amyloid | | Familial Mediterranean fever, |
| A protein (ApoSAA) | | predominant |
| renal involvement (autosomal | | |
| recessive) | | |
| AA derived from Serum amyloid | | Muckle-Well's syndrome, |
| A protein (ApoSAA) | | nephropathy, |
| | | deafness, urticaria, limb pain |
| Unknown | | Cardiomyopathy with persistent atrial |
| | | standstill |
| Unknown | | Cutaneous deposits (bullous, papular, |
| | | pustulodermal) |
| AH amyloid protein, derived | Aγ I | Myeloma associated amyloidosis |
| from immunoglobulin heavy | | |
| chain (gamma I) | | |
| ACal amyloid protein from | (Pro)calcitonin | Medullary carcinomas of the thyroid |
| (Pro) calcitonin | | |
| AANF amyloid protein from | | Isolated atrial amyloid |
| atrial natriuretic factor | | |
| Apro from Prolactin | | Prolactinomas |
| Abri/ADan from ABri peptide | | British and Danish familial Dementia |

Data derived from Tan SY, Pepys MB. Amyloidosis. Histopathology, 25(5), 403-414 (Nov 1994), WHO/IUIS Nomenclature Subcommittee, Nomenclature of Amyloid and Amyloidosis. Bulletin of the World Health Organisation 1993; 71: 10508; and Merlini et al., Clin Chem Lab Med 2001; 39(11): 1065-75.

All forms of amyloidosis are characterized by the deposition of fibrils in various tissues. The rigid, linear and nonbranched amyloid fibrils are the result of the misfolding of a protein into a β-pleated sheet configuration.

Next to diseases associated with amyloidosis, the present invention is also applicable for the treatment of biofilms. Biofilms are structured communities of microbial species embedded in a biopolymer matrix on either biotic or abiotic substrates (Davey et al. Microbiol. Mol. Biol. Rev. 64:847-867, 2000; Zobell et al., J. Bacteriol. 46:39-59, 1943). These slow-growing populations of bacteria focus on perseverance, as opposed to free-floating planktonic bacteria, and are present in virtually all natural and pathogenic ecosystems (Costerton et al., Ann. Rev. Microbiol. 41:435-464, 1987). On the one hand, beneficial properties of biofilms have been put to use in industrial processes, but on the other hand they pose substantial challenges including chronic biofilm-related infections (Characklis et al., Adv. Appl. Microbiol., 29:93-137, 1983; Eisenmann et al., Appl. Environ. Microbiol. 67:4286-4292, 2001). Most importantly, the biofilm is characterized by its resistance to biocides, antibiotic chemotherapy, and clearance by humoral or cellular host defense mechanisms (Costerton et al., Science 284:1318-1322, 1999; Dunne et al., Clin. Microbiol. Rev. 15:155-166, 2002). Therefore, treatments with traditional concentrations of biocides or antibiotics are ineffective at eradicating the biofilm populations.

Biofilm formation is a complex dynamic process. Surfaces are normally conditioned with water, lipids, albumin, extracellular polymer matrix, or other nutrients from the surrounding environment. Once bacteria arrive at the surface, different physical, chemical and biological processes take place and bacteria adhere to the surface, initially in a reversible association. On the abiotic surface, this primary attachment is generally mediated by non-specific interactions such as electrostatic, hydrophobic or van der Waals forces, whereas adhesion to biotic surfaces occurs through specific molecular docking mechanisms. Planktonic cells are thought to initiate the contact with a surface either randomly or in a directed fashion via chemotaxis and motility. Studies have shown that the rate of bacterial adhesion to a wide variety of surfaces is affected by some physical characteristics such as hydrophobicity of surfaces (Carpentier et al., J. Appl. Bacteriol. 75:499-511, 1993; Fletcher et al., Appl. Environ. Microbiol. 37:67-72, 1979). Other studies suggest that motility is very important for the planktonic cells to make initial contacts with an abiotic surface, and for bacteria to spread across the surface (O'Toole et al., Mol. Microbiol. 30:295-304, 1998; Pratt et al., Mol. Microbiol. 30:285-293, 1998). Flagella-mediated motility can bring the cell within close proximity of the surface to overcome repulsive forces between bacterium and the surface where bacterium will be attached. Bacteria form biofilms preferentially in very high-shear environments as compared with low shear environments (Donlan et al., Clin. Microbiol. Rev. 15:167-193, 2002). One of the explanations is that the high-shear flow aids in organization and strengthens the biofilm, making it more resistant to mechanical breakage.

After binding to the surface, bacterial cells start the process of irreversible adhesion through exopolymeric matrix, proliferation, and accumulation as multilayered cell clusters. These extracellular matrices, composed of a mixture of materials such as polysaccharides, proteins, nucleic acids, and other substances, are considered to be essential in cementing bacterial cells together in the biofilm structure, in helping to trap and retain nutrients for biofilm growth, and in protecting cells from dehydration and the effects of antimicrobial agents (Boyd et al., Appl. Environ. Microbiol. 60:2355-2359, 1994; Davies et al., Appl. Environ. Microbiol. 61:860-867, 1995).

Once having irreversibly attached to a surface, bacterial cells undergo phenotypic changes, and the process of biofilm maturation begins. Bacteria start to form microcolonies either by aggregation of already attached cells, cell division or cell recruitment of planktonic cells or cell flocs from the bulk liquid. The attached cells generate a large amount of extracellular components which interact with organic and inorganic molecules in the immediate environment to create the glycocalyx. The microcolony is the basic unit of biofilm growth (Costerton et al., Annu. Rev. Microbiol. 49:711-745, 1995). Mature biofilms consist of mushroom-like microcolonies with intervening water channels, which serve as transport channels for nutrients and metabolic wastes (Rasmussen et al., Biotechnol. Bioeng. 59:302-309, 1998). There are many microenvironments within a biofilm—each varying because of differences in local conditions such as nutrient availability, pH, oxidizing potential (redox), and so on. Cells near the surface of the biofilm microcolony are exposed to high concentrations of O₂, while near the center oxygen is rapidly depleted to near anaerobic levels (Lewandowski et al., Biofouling and Biocorrosion in Industrial Water Systems. Florida: Lewis, 175-188, 1994). The steep oxygen gradients are paralleled by gradients for either nutrients or metabolites from the biofilm, which creates a heterogenic environment even for the single-species of biofilm (de Beer et al., Biotechnol. Bioeng. 43:1131-1138, 1994; Walters et al., Antimicrob. Agents Chemother. 47:317-323, 2003).

It is now well established that bacterial cells communicate through the secretion and uptake of small diffusible molecules. Recent evidence indicates that biofilm formation might be regulated at the level of population density-dependent gene expression controlled by cell-to-cell signaling, or quorum sensing (Davies et al., Science 280:295-298, 1998). A large number of bacterial species are known to possess this communication mechanism, through which bacteria can sense changes in their environment and coordinate gene expression in favor of the survival for the entire community (Shirtliff et al., Chem. Biol. 9:859-871, 2002). These cell-cell communication systems regulate various functions as diverse as motility, virulence, sporulation, antibiotic production, DNA exchange, and development of multicellular structures such as biofilm and fruiting body formation (De Kievit et al., Medical Implications of Biofilms. Cambridge: University Press, 18-35, 2003; Hentzer et al., Microbial biofilms. Washington, D.C.: ASM Press, 118-140, 2004; Smith et al., J. Food Prot. 67:1053-1070, 2004).

Biofilms and especially the increased resistance against antibiotics and desinfectants of the bacteria comprised in the biofilms pose an important problem for medicine and industry. Biofilms are associated with more than 65% of all medical infections (Centers for Disease Control and Prevention, Atlanta, Ga.).

Indwelling Medical Device Infections

With the aging of patient populations and increases in chronic diseases, indwelling medical devices (IMD) such as catheters, joint prostheses, pacemakers and heart valves, have become a significant part of medical practice (Donlan et al., Emerg. Infect. Dis. 7:277-281, 2001; Weinstein et al., Infectious Diseases Conference summaries, Medscape, Inc., 2000). Microorganisms are capable of forming biofilms on these surfaces. The classic characteristics of biofilm-related infections of IMD include the following: (a) biofilms form on an inert surface or dead tissue, (b) they grow slowly with a delayed onset of symptoms, (c) biofilm infection is not resolved by host defense mechanisms, (d) planktonic cells released from biofilms (programmed detachment) act as a nidus of infection, and (e) antibiotic therapy does not kill mature biofilms (Costeron et al., Annu. Rev. Microbiol. 49:711-745, 1995). The ability of biofilms to evade the host immune responses and their enhanced antimicrobial resistance phenotype make biofilm-related IMD infections very difficult to manage (Mukherjee et al., Infect. Immun. 71:4333-4340, 2003; Kuhn et al., Antimicrob. Agents Chemother. 46:1773-1780, 2002; Kuhn et al., Curr. Op. Infect. Dis. 5:186-197, 2004). Often, the only reliable treatment for biofilm-associated IMD infections is the removal of the infected device. However, this can be associated with increased morbidity and mortality, prolonged hospitalization, and increased healthcare costs (Donlan et al., Clin. Infect. Dis. 33:1387-1392, 2001).

Dental Plaque

The oral cavity is home to ca. 700 bacterial species, many of which engage in biofilm formation through a sequential and ordered accumulation on tooth surfaces (Bjarnsholt et al., in Biofilm Infections, Springer New York, chap. 4, 2011). Supra- and subgingival oral biofilms are complex and dynamic multispecies communities even at very early stages when only a few cells are present. The arrangement of cells is influenced by specific cell-cell recognition which establishes particular combinations of bacteria. Inter-bacterial communication is likely to play a significant role in metabolism within these communities. In its early stages of development, plaque is dominated by streptococci and actinomyces, and it exists in commensal harmony with the host. However, in the absence of adequate oral hygiene, ecological shifts occur within the microbial community which initiate two complex oral diseases: caries and periodontal diseases. These diseases do not arise from sudden infection by, or even chronic presence of, a single pathogen—instead they result from over-representation of several pathogenic bacteria that are typically found in low numbers in the healthy commensal plaque microflora and the concomitant under-representation of the normally more abundant commensal organisms. Dental caries (tooth decay that ultimately leads to cavitation) result from ecologically controlled metabolic processes in the plaque biofilm that disturb mineral balance at the biofilm—tooth interface. Comprehensive epidemiological studies show that caries and subsequent periapical infections remain the single most important reason for tooth extractions worldwide, even greater than the number for periodontal disease (Baelum et al., in: Dental caries: the disease and its clinical management, 2^{nd} edn. Blackwell Munksgaard, Oxford, 2008). Periodontal diseases are complex polymicrobial biofilm infections that involve interactions between a large subset of oral bacteria and the host. Accumulation of plaque at the gingival margin triggers an inflammatory reaction (gingivitis). In susceptible subjects, the inflammatory process expands deeper into the tissues resulting in the loss of collagen fibers that support the tooth (attachment loss), as well as the resorption of alveolar bone: the two hallmarks of periodontitis.

Food Industry

Food and food processing surfaces are susceptible to colonization by food-borne microorganisms. The formation of biofilms by pathogenic or spoilage microorganisms has a considerable negative impact on food safety and quality (Blaschek et al., Biofilms in the food environment, Blackwell Publishing, Oxford, 2007). Listeria monocytogenes is one of the important food-borne pathogens that cause severe illness (listeriosis) particularly among the young, old and immuno-compromised people. It is usually found in dairy products and can grow in a wide range of temperatures from 1 to 45° C., and at broad acidity levels from pH 4 to 9. This versatility enables L. monocytogenes to outcompete other bacteria in extreme conditions. It is quite dangerous to foods that are stored at refrigerator temperatures, because these psychrophilic bacteria can multiply and thrive during storage. Due to the enhanced antibiotic and stress resistance, bacteria in biofilms can survive various stress conditions that are commonly used in food processing. They tend to maintain themselves in niches in the processing environment, particularly in hard to clean areas such as drains and crevices and cracks in surfaces or seals (Tompkin et al., J. Food Prot. 65:709-725, 2002). An individual persistent strain can sometimes be repeatedly isolated from the same facility over a period of months or years (Kathariou et al., J. Food Prot. 57:720-724, 2002).

Corrosion

The term microbiologically influenced corrosion (MIC) is used to designate corrosion due to the presence and activities of microorganisms. This corrosion occurs in environments that can support the growth of microorganisms, including environments where corrosion would not be predicted, and the rates can be exceptionally high. Microbiologically influenced corrosion has been documented in chemical, food, and pulp and paper processing; conventional and nuclear power generation; exploration, production, transportation, storage, and use of hydrocarbon fuels; and marine industries and fire protection systems (Little et al., Microbiologically influenced corrosion, Wiley, N.J., 2007). Microbiologically influenced corrosion is reported to account for 20% of the total cost of corrosion (Flemming et al., Microbiologically influenced corrosion of materials, Springer-Verlag, New York, 1996). There are numerous mechanisms and causative organisms for MIC that can vary among metals and alloys and operating conditions for the same materials.
Ennoblement: Biofilm formation on passive metals can shift the open-circuit corrosion potential in the noble direction, and produce accompanying increases in current density.
Oxygen concentration cells: Respiring aerobic microbial cells on metal surfaces can result in local anodes and cathodes and formation of differential aeration cells. Under aerobic conditions, areas under respiring colonies become anodic and surrounding areas become cathodic.
Metal concentration cells: Microorganisms that colonize metal surfaces produce extracellular polymeric substances (EPS) and form a gel matrix on the metal. In general, EPS are acidic and contain functional groups that bind metals. Metal ions concentrated from the aqueous phase or from the substratum into the biofilm can increase corrosion rates by providing an additional cathodic reaction.
Inactivation of corrosion inhibitor: Biofilms reduce the effectiveness of corrosion inhibitors by creating a diffusion barrier between the metal surface and the inhibitor in the bulk medium. Aliphatic amines and nitrites used as corrosion inhibitors can be degraded by microorganisms, decreasing the effectiveness of the compounds and increasing the microbial populations.
Reactions within biofilms: Reactions within biofilms are generally localized, affecting mechanisms and accelerating rates of electrochemical reactions leading to corrosion.
While corrosion influencing reactions may be attributed to a single group of organisms, the most aggressive MIC occurs with natural populations containing many types of microorganisms.

Inherent resistance of biofilm bacteria to antibiotics and biocides is a growing problem in medicine and industry. The current evolution in medicine with increasing use of antibiotics favors the growth of bacteria in colonies with an augmented antibiotic resistance. The increased use of indwelling medical devices results in more frequent occurrence of infections related to biofilms in the body. Biofilms may exhibit antibiotic resistance three or more orders of magnitude greater than those displayed by planktonic bacteria of the same strain depending on the species-drug combination (Ceri et al., J. Clin. Microbiol. 37:1771-1776, 1999). After exposure to antibiotics, a small surviving population of persistent bacteria can repopulate the surface immediately, and become more resistant to further antibiotic treatment. Paradoxically, once dispersed from the biofilm, those bacterial cells typically revert to an antibiotic susceptible form. A number of factors have been considered for the resistance of biofilms:
1. Slow penetration of antibiotics The biofilm extrapolymeric matrix has the potential to reduce the penetration of antibiotics and biocides either by physically slowing diffusion or chemically reacting with these compounds. Exopolysaccharides act as an ion exchanger, and sequester hydrophilic and positively charged antibiotics such as aminoglycosides.
2. Slow growth rate of biofilm cells Also oxygen limitation and low metabolic activity in the biofilm interior contributes to the antibiotic tolerance (Walters et al., Antimicrob. Agents Chemother. 47:317-323, 2003). Bacterial cells in biofilms constitute a heterogeneous population with varied growth rates in different compartments of the biofilm, depending on the limitation of nutrients. Slow growing cells generally have a much reduced metabolic activity, resulting in reduction of antimicrobial susceptibility.
3. Increased rate of genetic transfer in the biofilm Bacteria may acquire antibiotic resistance through either horizontal gene transfer (such as genes encoded on plasmid, transposon, or integron) or through mutation in different chromosomal loci. Since most bacteria found in nature live in biofilms on surfaces or at interfaces, it is likely that gene transfer by conjugation plays an important role for spreading antibiotic resistance among different bacterial species. Biofilms are ideally suited to the exchange of genetic material of various origins due to the close contact and relative spatial stability of bacteria within biofilms.
4. Expression of resistance genes in biofilms Sub-lethal concentrations of antibiotics select resistance in microorganisms (Khachatourians et al., CMAJ 159:1129-1139, 1998). The varied antibiotic concentrations in the different compartments of the biofilm may exert different selective pressure on the biofilm bacteria. In considering the heterogeneous populations inside the biofilm due to different concentrations of oxygen, nutrients, pH and other environmental factors, biofilms cells are expected to possess different levels of antibiotic resistance.
5. Hypermutation in biofilms The high antibiotic resistance of biofilms may be explained in part by the hypermutation phenomenon as observed in stressed bacterial cells. Both environmental and physiological stress conditions, such as starvation and antibiotic treatment, can transiently increase the mutation rates in sub-populations of bacteria allowing the bacteria to evolve faster (Velkov et al., J. Biosci 26:667-683, 2001; Blazquez et al., Clin. Infect. Dis. 37:1201-1209, 2003). Approximately 1% of pathogenic E. coli and Salmonella isolates from both food-related outbreaks of disease and the natural environment, and 20% of Pseudomonas isolates from the lungs of cystic fibrosis patients are strong mutators with very high mutation rates (LeClerc et al., Science 274:1208-1211, 1996; Oliver et al., Science 288:1251-1253, 2000). These hypermutable strains are mainly defective in methyl-directed mismatch repair genes, a DNA repair and error-avoidance system.
6. Multicellular nature of the biofilm community Bacteria within a biofilm display many types of phenotypes, with broad metabolic and replicative heterogeneity, providing the community as a whole with enormous capability to resist stresses, whether from host defense systems or antimicrobial agents (Stoodley et al., Annu. Rev. Microbiol. 56:187-209, 2002). Biofilm cells have been recognized as multicellular organisms, using the sophisticated signal transduction networks to regulate gene expression and cell differentiation (Shapiro et al., Annu. Rev. Microbiol. 52:81-104, 1998; Stewart et al., Trends Microbiol. 9:204, 2001). This multicellular behavior permits biofilm cells to efficiently utilize resources for cell growth and provide collective defense against clearance by humoral or cellular host defense mechanisms and killing by biocides or antibiotic chemotherapy.

Currently, strategies for biofilm treatment include chemical elimination of bacteria with antibiotics and biocides, prevention of bacterial cell adhesion to the substrate, reduction of polysaccharide production and disruption of cell-to-cell communication involved in biofilm formation through physical, chemical and biological approaches.

In industrial applications, peracetic acid and chlorine are considered as the most efficient disinfectants to remove biofilms from surfaces. However, the effect is only obtained if disinfectants are used at high concentrations for long reaction times and after pre-treatment with detergent (Jessen et al., Internat. Biodeterioration and Biodegradation 51:265-269, 2003; Chmielewski et al., Comp. Rev. Food Sci. Food Safety 2:22-32, 2003). As expected, bacteria in suspension are more sensitive to disinfectants than bacteria in biofilms (Harkonen et al., Wat. Sci. Technol. 39:219-225, 1999; Stopforth et al., J. Food Prot. 20:2258-2266, 2003). Among the disinfectants tested, peracetic acid is more effective than the aldehydes, hydrogen peroxide, or chlorine against biofilm bacteria (Exner et al., Zbl. Bakteriol. Hyg. B 183:549-563, 1987). Bacteria can become resistant to these biocides as well, and to avoid a buildup of resistant pathogens rotation of disinfectants should be considered.

In medicine, a combination of various treatments has been tested for controlling biofilm formation. These can include combinations of antibacterial agents such as the common practice of administering rifampin with another antibacterial, or the combination of both chemical and physical intervention (Rediske et al., Antimicrob. Agents Chemother. 43:1211-1214, 1999; Soboh et al., Antimicrob. Agents Chemother. 39:1281-1286, 1995). Raad et al. reported that the combination of minocycline and rifampin was highly effective in preventing the colonization of catheters with slime-producing S. epidermidis and S. aureus (Raad et al., s. Antimicrob. Agents Chemother 39:2397-2400, 1995). Both in vitro and in vivo (animal model) studies have indicated that low-frequency and low power-density ultrasound combined with aminoglycoside antibiotics significantly reduce E. coli biofilms (Rediske et al., J. Gen. Appl. Microbiol. 44:283-288, 1998). Combination of low electrical currents with antibiotic treatment also appears more effective in controlling biofilms by increasing the diffusion of charged molecules and antibiotics through the biofilm matrix (Costerton et al., Antimicrob. Agents Chemother. 38:2803-2809, 1994; Davis et al., Antimicrob. Agents Chemother. 36:2552-2555, 1992). However, it is currently unknown whether all of these approaches can be utilized in the patient.

Since no single method or chemical completely eliminates biofilm microorganisms, rigorous care has to be taken to control biofilm formation. Both in industrial and medical environments it is recommended that various preventive and control strategies be implemented such as hygienic layout, design of equipment, choice and coating of materials, correct use and selection of detergents and disinfectants and appropriate sanitation practices.

Recent investigations have identified amyloid fibrils as a constituent of the biofilm extracellular matrix. The amyloid fibrils are part of the bacterial surface structures that are important for adhesion and later development of mature biofilm. Larsen et al. applied staining methods using Thioflavin T and conformationally specific antibodies that target amyloid fibrils to stain amyloid adhesions, and combined this with methods for simultaneous identification of the bacteria directly in the biofilms (Larsen et al., Env. Biol. 9:3077-3090, 2007). This allowed an in situ detection and quantification of bacteria expressing amyloid adhesions in natural biofilms. The results demonstrate that amyloid adhesins were indeed very common in natural biofilms, and that the diversity and quantity of prokaryotes producing amyloid structures in natural biofilm were high in several environmental habitats. Romero et al. demonstrated that the protein TasA, one of the main components of the extracellular matrix of bacillus subtilis biofilms, forms fibrils of variable length and 10-15 nm in width (Romero et al., PNAS 107:2230-2234, 2010). Biochemical analyses, in combination with the use of specific dyes and microscopic analyses indicated that TasA forms amyloid fibrils that are essential in the formation of robust biofilms. Consistent with the stable nature of amyloid fibrils, TasA fibers require harsh treatment (e.g., formic acid) to be depolymerized.

The present invention proposes a method to disrupt the structural stability of amyloid fibrils and can be applied to target both the amyloid fibrils associated with amyloid diseases, as the amyloid fibrils that form a structural component of biofilms.

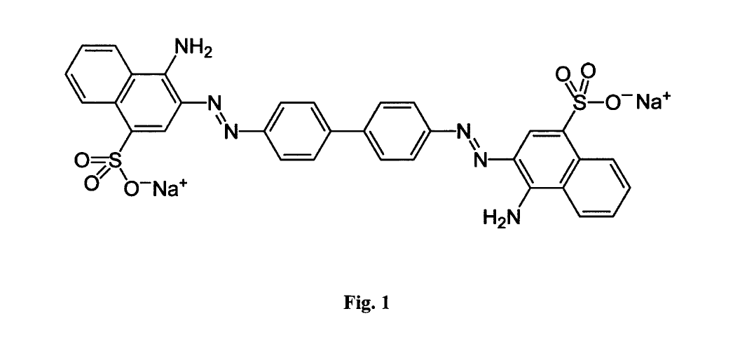

Dye Congo red (CR) is the sodium salt of benzidinediazo-bis-1-naphthylamine-4-sulfonic acid (FIG. 1) which is a water soluble diazo dye. Due to the small molecular size (696.66 g/mol) and penetrability through the blood-brain barrier, Congo red and congeners can be used for post-mortem and in vivo visualization and quantification of brain amyloids.

While Congo red has an affinity for binding to fibril proteins enriched in β-sheet conformation, the dye intercalates between the β-strands of the fibrils. In vitro research has been focused on amyloid fibrils derived from the insulin hormone, since amyloid fibrils from cerebral tissue are hard to obtain. Insulin has the ability to form amyloid-like β-sheet fibrils under the specific conditions of low pH and elevated temperature and serves as a model system for the pathological amyloid fibrils (D. Waugh, J. Am. Chem. Soc. 68, 247-250 (1946)). Infrared, Raman and UV-visible spectroscopic techniques reveal that the interaction of Congo red (CR) is similar in brain tissue and in amyloid fibrils obtained from insulin (J. Sajid et al., Journal of Molecular Structure 408/409, 181-184 (1997)). The intercalation mechanism involves the disruption of the four main-chain hydrogen bonds between the two β-strands that are tethered with each other through new hydrogen bonds with the Congo red nitrogen atoms (D. B. Carter and K.-C. Chou, Neurobiology of Aging 19, 37-40 (1998)). The Carter-Chou model postulates the alignment of CR parallel to the peptide chain and perpendicular to the axis of the fibril. In this way the dye molecule is sandwiched between the β-strands of the fibrils. Infrared and Raman spectroscopy studies indicate that the sulfonic acid oxygen groups (SOD of the dye form an electrostatic bond with a basic amino acid side chain. The aggregation of the amylin hormone in Type-2 diabetes generates fibrous depositions in the pancreatic β-cells. These fibrillar structures are identified as amyloidogenic and are made up of extended β-strands that run perpendicular to the fibril axis (O. S. Makin and L. C. Serpell, J. Biol. Chem. 335, 1279-1288 (2004)). Although fibrils may be composed of different proteins linked to different diseases, they share common structural features.

Our work demonstrates that binding an intercalating molecule with a negatively charged group such as CR is essential to dismantle the amyloid fibrils into soluble proteins. The fact that CR binds to preformed β-amyloid fibrils in such a way that it is sandwiched between the monomers of the fibril is crucial to destabilize the conformation of the stained fibril in a second binding process.

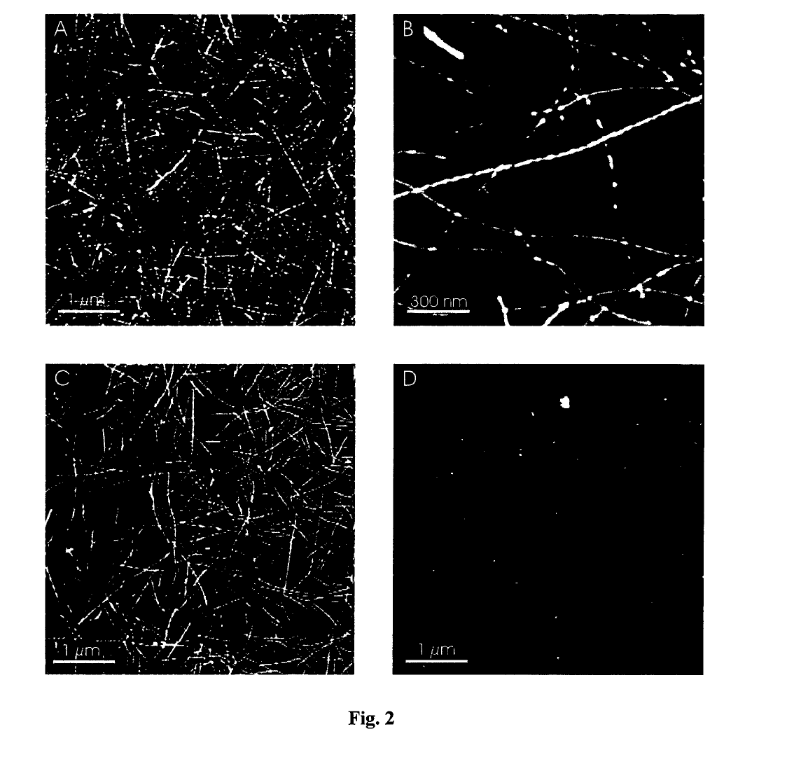

Amyloid fibrils from the insulin hormone were obtained by dissolving insulin (3.5 mmol/L) in water adjusted to pH 2 with HCl and heated to 70° C. for 2 hours. Samples were diluted to a concentration of 7 μmol/L at room temperature and transferred to a piranha (4:1, H₂SO₄:H₂O₂) cleaned naturally oxidized silicon wafer. After 15 minutes of incubation the silicon surface was rinsed with water. The amyloid fibers on the silicon surface are then incubated with a 140 μmol/L solution of CR in water during 2 hours and successively rinsed with water. FIG. 2 shows atomic force microscopy (AFM) images of amyloid fibrils from insulin before incubation with CR (A and B) and after incubation with CR(C). The average cross section of the dye-amyloid fiber has the same dimension as in the absence of CR, suggesting that the binding of CR is an intercalating process that only elongates the fibrils.

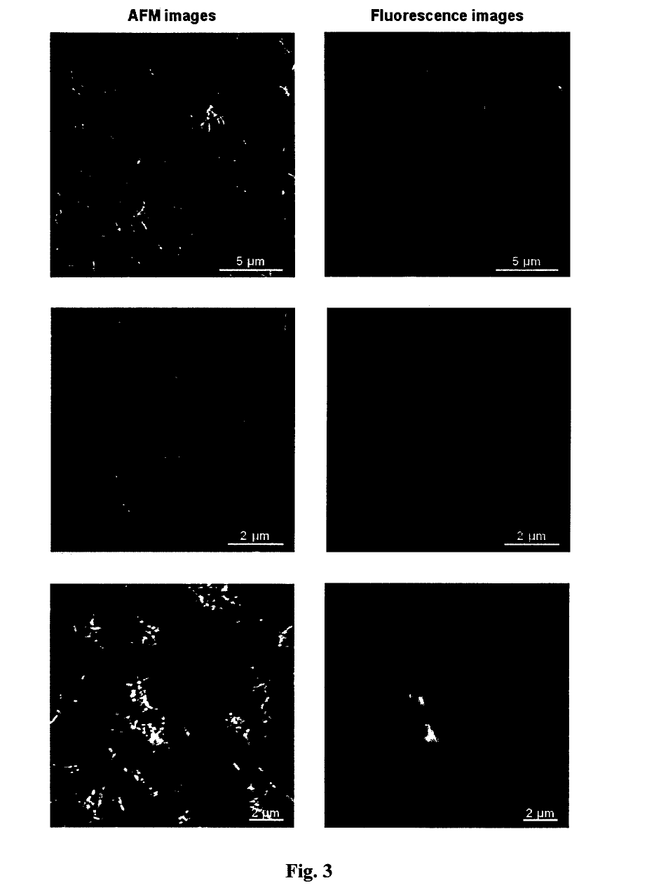

The dye-amyloid fibril complexes were also imaged with fluorescence microscopy, as CR is a fluorophore. FIG. 3 shows atomic force microscopy images (left panels) of insulin fibrils incubated with CR and fluorescence microscopy images (right panels) of the same fibrils. Bright contrast in the fluorescence images corresponds to the presence of the fluorophore on the insulin fibrils, which themselves are not fluorescent. It is clear that the CR is bound or intercalated evenly in the insulin fibrils.

FIG. 2 D) shows a dye-amyloid sample incubated with a 5 mmol/L solution of silver ions during 15 minutes and successively rinsed with water and dried under a flow of nitrogen gas. The AFM image reveals the total disintegration of the dye-amyloid fibers into a small number of sparsely distributed particles over the silicon surface. The heights of the particles (1-2 nm) is comparable to the 1.1 nm height of native insulin monomers and dimers (1.49 nm) (Ahmad et al., Biochemistry 42, 11404-11416 (2003); R. Jansen et al., Biophys. J. 88, 1344-1353 (2005)), indicating that the fibers are disintegrated up to the level of monomers. The predominant particles are soluble dimers and higher order aggregates with the hydrophobic regions sequestered from the aqueous environment (D. Watson et al., Neurological Research 27, 869-881 (2005)). The soluble oligomeric amyloid-beta peptides are the principal constituent of amyloid deposits such as plaques and intraneuronal tangles.

The AFM measurements reveal that the intercalated CR destroys the amyloid fibrils in the presence of silver or other metal ions (not shown), resulting in the total disintegration of the amyloid fibrils into peptide monomers. The positively charged metal ions bind to the negatively charged sulfonate groups of the dye and interfere with the electrostatic bond between the negatively charged group of the CR molecule and the basic amino acid side chain of the fibril. This way the dye-peptide bond is disrupted at the monomer level, which causes the disintegration of the fibril. Our in vitro study points out that neurodegenerative disorders can be treated by negatively charged molecules, like CR which intercalate between the amyloid β-strands and induce a destabilization of the CR-fibril complex when metallic ions such as silver ions are introduced. Similar results are obtained using copper(I), gold(I), palladium and lead ions and metallic colloids of silver and gold (picture not shown).

The magnitude of the therapeutic or prophylactic dose of the compounds of the present invention in the treatment or prevention of T2DM, pathological consequences of T2DM, inhibition of amyloidosis and prevention of pancreatic β-cell death depends upon severity and nature of the condition being treated and the route of administration. The dose and the frequency of the dosing will also vary according to age, body weight and response of the individual patient. The metal ion such as silver, gold(I), copper(I), palladium or lead ions or a metal colloid of silver or gold is hereby preferably administered after the negatively charged planar aromatic molecule, for instance diazo dye and preferably when excess of the negatively charged planar aromatic molecule, for instance diazo dye, is removed from the subject (mammalian patient) for instance by natural excretion.

In general the total daily dose range for a compound of the present invention is from approximately 0.1 to approximately 500 mg in single or repeated doses.

Any suitable routes of administration may be employed to provide an effective dosage of the compounds of the present invention. Possible routes are not limited by oral, intravenous, topical and parenteral administrations, with oral administration representing a preferred route.

Compounds of the present invention may be administered in association with one or more inert carriers, excipients and diluents forming a pharmaceutical composition. Certain preferred oral compositions contain between approximately 0.1% and approximately 75% of compounds of formulas I.

Solid compositions for oral administration may include binders, such as syrups, acacia, sorbitol, polyvinylpyrrolidone, carboxymethylcellulose, ethyl cellulose, microcrystalline cellulose or gelatin and mixtures thereof: excipients, such as starch, lactose or dextrins; disintegrating agents, such as alginic acid, sodium alginate, primogel and the like; lubricant, such as magnesium stearate, heavy molecular weight acids such as stearic acid, high molecular weight polymers such as polyethylene glycol; sweetening agents, such as sucrose or saccharine; flavoring agents, such as peppermint, methyl salicylate or orange flavoring; and coloring agents.

The liquid pharmaceutical compositions of the Invention, whether they are solutions, suspensions or other like form, may Include sterile diluents such as water for injection, saline solution, preferably physiological saline, Ringer's solution, or isotonic sodium chloride, fixed oils such as synthetic mono or diglycerides which may serve as the solvent or suspending medium, polyethylene glycols, glycerin, propylene glycol or other solvents. Inhibition of amyloidosis and prevention of cell degeneration or death depends upon severity and nature of the condition being treated and the route of administration.

### DRAWING DESCRIPTION

### Brief Description of the Drawings

The present invention will become more fully understood from the detailed description given herein below and the accompanying drawings which are given by way of illustration only, and thus are not limitative of the present invention, and wherein:

FIG. 1 Molecular structure of the diazo dye Congo red.

FIG. 2 Atomic force microscopy image of amyloid fibrils from insulin: A and B) prior to incubation with Congo Red, C) after incubation with Congo Red and D) after incubation of the Congo Red-amyloid fibril complex with a solution of 5 mmol/L silver ions.

FIG. 3 Atomic force microscopy (left panels) and corresponding fluorescence microscopy images (right panels) of insulin fibrils incubated with Congo Red.

What is claimed is:

1. A pharmaceutical composition comprising a pharmaceutically acceptable carrier and the substances (i) a sodium salt or any pharmaceutically acceptable salt of benzidinediazo-bis-1-naphthylamine-4-sulfonic acid or of a pharmaceutically acceptable derivative of benzidinediazo-bis-1-naphthylamine-4-sulfonic acid and (ii) a metal ion.

2. The pharmaceutical composition according to claim 1, comprising and (i) benzidinediazo-bis-1-naphthylamine-4-sulfonic acid or a sodium salt or any pharmaceutically acceptable salt thereof and (ii) a metal ion and a pharmaceutically acceptable excipient.

3. The pharmaceutical composition according to claim 1, comprising and (i) disodium 3,3′-[[1,1′-biphenyl]-4,4′-diylbis(azo)]bis(4-aminonaphthalene-1-sulphonate) and 2) a metal ion and a pharmaceutically acceptable excipient.

4. The pharmaceutical composition according to claim 1, wherein the benzidinediazo-bis-1-naphthylamine-4-sulfonic acid salt is Congo red.

5. The pharmaceutical composition according to claim 1, wherein the metal ion is a positive ion.

6. The pharmaceutical composition according to claim 1, wherein the metal ion is silver.

7. The pharmaceutical composition according to claim 1, wherein metal ion is an ion of the group consisting of silver, gold(I), copper(I), palladium, lead, zinc, metal colloid of silver and metal colloid of gold.

8. The pharmaceutical composition according to claim 1, comprising and (i) dipotassium 3,3′-[[1,1′-biphenyl]-4,4′-diylbis(azo)]bis(4-aminonaphthalene-1-sulphonate) and (ii) a metal ion and a pharmaceutically acceptable excipient.

9. The medical or pharmaceutical composition of any one of the previous claims, that further comprises an agent that modifies the release of the substance, a glidant/diluent, a filler, a binder/disintegrant, a lubricant, a subcoat, a topcoat, an enteric coat, and any combination thereof.

10. The pharmaceutical composition according to claim 1, wherein the substances are present in an amount sufficient to inhibit cellular toxicity induced by amyloid, and a pharmaceutically acceptable vehicle.

11. The pharmaceutical composition according to claim 1, wherein the substances are present in an amount sufficient to inhibit amyloidosis induced neurodegeneration.

12. The pharmaceutical composition according to claim 1, wherein the substances are present in an amount sufficient to destabilize amyloid fibril deposits.

13. The pharmaceutical composition according to claim 1, wherein the substances are present in an amount sufficient to destroy amyloid fibril deposits.

14. The pharmaceutical composition according to claim 1, wherein the substances are present in an amount sufficient to treat amyloidosis and prevent death of beta-cells in type 2 diabetes mellitus.

15. The pharmaceutical composition according to claim 1, wherein the amyloid-related disease is cerebral amyloid angiopathy.

16. The pharmaceutical composition according to claim 1, wherein the amyloid-related disease is Alzheimer's disease.

17. The pharmaceutical composition of any one of the previous claims, formulated for oral administration.

18. A pharmaceutical pack comprising the substances and (i) a sodium salt or any pharmaceutically acceptable salt of benzidinediazo-bis-1-naphthylamine-4-sulfonic acid or of a pharmaceutical acceptable derivative of benzidinediazo-bis-1-naphthylamine-4-sulfonic acid and (ii) a metal ion.

19. The pharmaceutical pack according to claim 18, comprising and (i) benzidinediazo-bis-1-naphthylamine-4-sulfonic acid or a sodium salt or any pharmaceutically acceptable salt thereof and (ii) a metal ion and a pharmaceutically acceptable excipient.

20. The pharmaceutical pack according to claim 18, comprising and (i) disodium 3,3′-[[1,1′-biphenyl]-4,4′-diylbis(azo)]bis(4-aminonaphthalene-1-sulphonate) and (ii) a metal ion and a pharmaceutically acceptable excipient.

21. The pharmaceutical pack according to claim 18 wherein the benzidinediazo-bis-1-naphthylamine-4-sulfonic acid salt is Congo red.

22. The pharmaceutical pack according to claim 18, wherein the metal ion is a positive ion.

23. The pharmaceutical pack according to claim 18, wherein the metal ion is silver.

24. The pharmaceutical pack according to claim 18, wherein metal ion is an ion of the group consisting of silver, gold(I), copper(I), palladium, lead, zinc, metal colloid of silver and metal colloid of gold.

25. The pharmaceutical pack according to claim 18, comprising (i) dipotassium 3,3′-[[1,1′-biphenyl]-4,4′-diylbis(azo)]bis(4-aminonaphthalene-1-sulphonate) and (ii) a metal ion and a pharmaceutically acceptable excipient.

26. The pharmaceutical pack of any one of the previous claims 18 to 25, wherein the substances (i) and (ii) are formulated separately and in individual dosage amounts.

27. The pharmaceutical pack of any one of the previous claims 18 to 25, wherein the substances (i) and (ii) are formulated together and in individual dosage amounts.

28. The composition or pack of any one of the previous claims 1 to 27; for use in a treatment of amyloid-associated diseases.

29. The composition or pack of any one of the previous claims 1 to 27; for use in a treatment to cure or to stabilize amyloid-associated diseases.

30. The composition or pack according to claim 29, wherein the amyloid-related disease or condition is treated prophylactically or therapeutically.

31. The composition or pack of any one of the previous claims 1 to 30; comprising pharmaceutically effective amount of the substances for use in a treatment of amyloid-associated diseases

32. The composition or pack of any one of the previous claims 1 to 30; for use in a treatment of an amyloid-associated disease is Alzheimer's disease.

33. The composition or pack of any one of the previous claims 1 to 30; for use in a treatment of an amyloid-associated disease wherein the amyloid-associated diseases comprises Type 2 diabetes mellitus, amyloid A (reactive), secondary amyloidosis, familial mediterranean fever, familial amyloid nephropathy with urticaria and deafness (Muckle-wells Syndrome), amyloid lambda L-chain or amyloid kappa L-chain (idiopathic, myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosises), AIAPP or amylin insulinoma, atrial naturetic factor (isolated atrial amyloid), procalcitonin (medullary carcinoma of the thyroid), gelsolin (familial amyloidosis (Finnish)), cystatin C (hereditary cerebral hemorrhage with amyloidosis (Icelandic)), AApo-A-I (familial amyloidotic polyneuropathy-lowa), AApo-A-II (accelerated senescence in mice), fibrinogen-associated amyloid; and Asor or Pr P-27 (scrapie, Creutzfeld Jacob disease, Gertsmann-Straussler-Scheinker syndrome, bovine spongiform encephalitis) and persons who are homozygous for the apolipoprotein E4 allele.

34. The composition or pack of any one of the previous claims 1 to 30; for use in a treatment of an amyloid-associated disease wherein the amyloid-associated diseases comprises a disease or condition selected from the group consisting of Alzheimer's disease, cerebral amyloid angiopathy, inclusion body myositis, macular degeneration, Down's syndrome, mild cognitive impairment, cognitive decline and hereditary cerebral hemorrhage.

35. The composition or pack of any one of the previous claims 1 to 30; for use in a treatment of disintegrating or destabilising accumulated amyloid material in the organs or tissues of the body.

36. The composition or pack of any one of the previous claims 1 to 35; wherein said the negatively charged secondary diazo dye or the negatively charged benzidinediazo-bis-1-naphthylamine-4-sulfonic is administered at 0.05-200 milligrams of substance.

37. The composition or pack of any one of the previous claims 1 to 36; wherein said the negatively charged secondary diazo dye or the negatively charged benzidinediazo-bis-1-naphthylamine-4-sulfonic is administered at 0.1-20 milligrams of substance.

38. The composition or pack of any one of the previous claims 1 to 36; wherein said metal cation is administered in a dosage amount equivalent to 0.05-1000 milligrams of negatively charged secondary diazo dye or the negatively charged benzidinediazo-bis-1-naphthylamine-4-sulfonic.

39. The composition or pack of any one of the previous claims 1 to 36; wherein said metal cation is administered in a dosage amount equivalent to 0.1-500 milligrams of negatively charged secondary diazo dye or the negatively charged benzidinediazo-bis-1-naphthylamine-4-sulfonic.

40. The composition or pack according to claims 28 to 30, for use in a treatment of amyloid-associated diseases wherein the amyloid-related disease is a disease associated with formation of a biofilm in a subject or patient in need of such treatment.

41. The composition or pack according to any one of the claims 28 to 30, for use in a treatment of amyloid-associated diseases wherein the amyloid-related disease is a disease associated with formation of a biofilm with amyloid fibrils in a subject or patient in need of such treatment.

42. The composition or pack according to any one of the claims 28 to 30, for use in a treatment of amyloid-associated diseases wherein the amyloid-related disease is a microbial infection or a sepsis.

43. The composition or pack according to any one of the claims 28 to 30, for use in a treatment of amyloid-associated diseases wherein the amyloid-related disease is a condition associated with a microbial infection or for decreasing bacterial growth in a animal subject or a human patient in need of such treatment.

44. The composition or pack according to any one of the claims 28 to 30, for use in a treatment of suppressing microbial biofilm growth wherever said suppression is desired.

45. The composition or pack according to any one of the claims 28 to 30, for use in a treatment of suppressing microbial biofilm formation and decreasing bacterial growth wherever said suppression is desired.

46. The composition or pack according to any one of the claims 28 to 30, for use in a treatment of amyloid-associated diseases wherein the amyloid-related disease is a disease associated with formation of a biofilm in a subject or patient in need of such treatment.

47. The composition or pack according to any one of the claims 28 to 30, wherein the amyloid-related disease is a condition associated with an infection in a subject or patient in need of such treatment a composition.

48. The composition or pack according to any one of the claims 28 to 30, wherein the amyloid-related disease is for increasing susceptibility to cytotoxic effects of antibacterial agents.

49. The composition or pack according to any one of the claims 28 to 30, wherein the amyloid-related disease wherein the patient has a wound selected from the group consisting of an ulcer, a laceration, a deep penetrating wound and a surgical wound.

50. The composition or pack according to any one of the claims 28 to 30, wherein the composition is selected from the group consisting of an oral tablet, capsule or liquid, a nasal aerosol, a throat wash, a mouth wash or gargle, a toothpaste, and a topical ointment.

51. The composition or pack according to any one of the claims 28 to 30, wherein the composition is selected from the group consisting of tampons, rinses, creams, and aerosols.

52. The composition or pack according to any one of the claims 28 to 30, wherein the composition is selected from the group consisting of soap, hair shampoo, toothbrushes, tooth paste, cotton swabs, antiperspirant, facial tissue, mouthwash, nail files, skin cleansers and toilet paper

53. The composition or pack according to any one of the claims 28 to 30, wherein the composition is a topical ointment, an irrigation solution or a component of a wound dressing.

54. The composition or pack according to any one of the claims 28 to 30, for use in a treatment by administering intravesicularly, topically, orally, rectally, ocularly, otically, nasally, parenterally, vaginally, intravenously, directly into an infected site, directly onto an indwelling prosthetic device or catheter.

55. The composition or pack according to any one of the claims 28 to 30, for reducing the risk of bacterial infection or sepsis in a person colonized with pathogenic bacteria, wherein said treatment occurs prior to said colonized person developing an illness due to said pathogenic bacteria.

56. The composition or pack according to any one of the claims 28 to 30, for use in a treatment for reducing the risk of bacterial infection or sepsis in a person colonized with pathogenic bacteria, wherein said treatment reduces the risk of bacterial infection or sepsis in said colonized person.

57. The composition or pack according to any one of the claims 28 to 30, for use in a treatment for reducing the risk of bacterial infection or sepsis in a person, wherein said person is an immunocompromised patient selected from the group consisting of leukemia patients, lymphoma patients, carcinoma patients, sarcoma patients, allogeneic transplant patients, congenital or acquired immunodeficiency patients, cystic fibrosis patients, and AIDS patients.

58. The composition or pack according to any one of the claims 28 to 30, for use in a treatment of inhibiting or preventing the formation of a biofilm or condition associated with formation of a biofilm on a biotic surface or in a biotic substrate.

59. The composition or pack according to claim 58, wherein the amyloid-related disease is a disease associated with formation of a biofilm in a subject or patient in need of such treatment and wherein the biofilm comprises more than one species of bacteria.

60. The composition or pack according to claim 58, wherein the amyloid-related disease is a disease associated with formation of a biofilm in a subject or patient in need of such treatment and wherein the biofilm comprises gram negative bacteria.

61. The composition or pack according to claim 58, wherein the amyloid-related disease is a disease associated with formation of a biofilm in a subject or patient in need of such treatment and wherein the biofilm further comprises gram positive bacteria.

62. The composition or pack according to claim 58, wherein the amyloid-related disease is a disease associated with formation of a biofilm in a subject or patient in need of such treatment and wherein the biotic surface or the biotic substrate is associated with bacterial infection.

63. The composition or pack according to claim 58, wherein the amyloid-related disease is a disease associated with formation of a biofilm in a subject or patient in need of such treatment and wherein the biotic surface or the biotic substrate is an epithelial or a mucosal layer.

64. The composition or pack according to claim 58, wherein the amyloid-related disease is a disease associated with formation of a biofilm in a subject or patient in need of such treatment and wherein the surface is an epithelial or mucosal surface of a mammal.

65. The composition or pack according to claim 58, wherein the amyloid-related disease is a disease associated with formation of a biofilm in a subject or patient in need of such treatment and wherein the biotic surface or the biotic substrate is a mucosa selected from the group consisting of mouth, vagina, astrointestinal tract and oesophageal tract.

66. The composition or pack according to claim 58, wherein the amyloid-related disease is a disease associated with formation of a biofilm in a subject or patient in need of such treatment and wherein the surface is the surface of a tooth.

67. The composition or pack according to claim 58, wherein the amyloid-related disease is a disease associated with formation of a biofilm in a subject or patient in need of such treatment and wherein the condition is an oral infection.

68. The composition or pack according to any one of the claims 27 to 67, wherein the substances are to be co-administered with one or more antibacterial agents.

69. The composition or pack according to any one of the claims 27 to 67, wherein the substance is to be co-administered with one or more antibacterial agents selected from the group consisting of antibiotics, antibodies, antibacterial enzymes, peptides, lantibiotics, lanthione-containing molecules and bacteriophages;

70. The composition or pack according to any one of the claims 27 to 67, wherein the substances are to be added in a single dose, in multiple doses, in multiple doses that are added on separate days, in multiple doses that are added on the same day, or are added continuously.

71. The composition or pack according to any one of the claims 27 to 67, wherein the microbial organism or microbe is a bacterium.

72. The composition or pack according to any one of the claims 27 to 67, for use in a treatment to reduce biofilm resistance
